(19) United States
(12) Patent Application Publication
(10) Pub. No.: US 2025/0288374 A1
(43) Pub. Date: Sep. 18, 2025

(54) METHOD AND APPARATUS FOR DRIVING SURGICAL INSTRUMENT

(71) Applicant: LivsMed Inc., Seongnam-si (KR)

(72) Inventor: Jin Hyuk YOON, Seongnam-si (KR)

(73) Assignee: LivsMed Inc., Seongnam-si (KR)

(21) Appl. No.: 19/003,074

(22) Filed: Dec. 27, 2024

(30) Foreign Application Priority Data
Mar. 13, 2024 (KR) 10-2024-0035293

(51) Int. Cl. A61B 34/30; A61B 34/00; A61B 34/20

(52) U.S. Cl. CPC A61B 34/30; A61B 34/20; A61B 34/25

(57) ABSTRACT

Provided is a method for driving a surgical instrument. The method includes: generating manipulation information of the surgical instrument based on an amount of change in a reference posture of a user input interface for controlling the surgical instrument; determining a target posture of the surgical instrument, corresponding to the generated manipulation information; generating target state information for at least one driving element based on whether the target posture exceeds a driving limit of the at least one driving element provided in the surgical instrument; and driving the at least one driving element according to the target state information.

### CROSS-REFERENCE TO RELATED APPLICATION

This application is based on and claims priority under 35 U.S.C. § 119 to Korean Patent Application No. 10-2024-0035293, filed on Mar. 13, 2024, in the Korean Intellectual Property Office, the disclosure of which is incorporated by reference herein in its entirety.

### BACKGROUND

### 1. Field

The present disclosure relates to a method and apparatus for driving a surgical instrument.

### 2. Description of the Related Art

In medical terms, surgery refers to the treatment of a disease by using medical devices to cut, slit, or manipulate skin, a mucous membrane, or other tissue. In particular, open surgery of cutting and opening the skin of a surgical site to treat, reshape, or remove organs therein causes bleeding, side effects, pain to a patient, and scars. Accordingly, recently, surgery using a robot or surgery performed by inserting only a medical device, for example, a laparoscope, a surgical instrument, a microsurgical microscope, or the like, in the body by forming a predetermined hole in the skin, has been spotlighted as an alternative.

Herein, a surgical robot refers to a robot that has a function of replacing a surgical action performed by a surgeon. The surgical robot may operate more accurately and precisely as compared with a human and enable remote surgery.

A surgical robot is generally composed of a master robot and a slave robot. When a surgical operator manipulates a control lever (for example, a handle) provided on the master robot, a surgical tool coupled to or held by a robot arm on the slave robot is manipulated to perform surgery.

However, some issues may arise due to performing the surgery by remotely manipulating the surgical tools through a surgical robot rather than physically manipulating the surgical tools directly by a surgical operator. For example, even though the surgical operator manipulates the control lever provided on the master robot, an issue may arise in which the slave robot does not perform the operation desired by the surgical operator due to mechanical constraints. When this mechanical constraint is reached, the control goal for realizing the posture of the surgical robot corresponding to the manipulation of the control lever is not calculated, so it takes more than a certain length of time to determine that control is not possible, which may cause an issue in which the operation of the surgical robot is interrupted. In addition, even when the surgical robot is driven within an operating limit, an issue may occur in which the surgical robot operates differently from the intuitive intention of a user.

The aforementioned background technology corresponds to technical information that has been possessed by the present inventor(s) in order to derive the present disclosure or which has been acquired in the process of deriving the present disclosure, and may not necessarily be regarded as well-known technology which had been known to the public prior to the filing of the present disclosure.

### SUMMARY

An aspect of the present disclosure is directed to providing a method and apparatus for driving a surgical instrument. In addition, an aspect of the present disclosure is directed to providing a computer-readable recording medium recording a program for executing the method on a computer.

The aspects of the present disclosure are not limited to those mentioned above, and other aspects and benefits not mentioned may be understood from the following description and may be more clearly understood by the embodiments of the present disclosure. In addition, the aspects and benefits to be solved by the present disclosure may be realized by the means indicated in the scope of claims and combinations thereof.

In accordance with an aspect of the present disclosure, a method for driving a surgical instrument may include: generating manipulation information based on an amount of change in a reference posture of a user input interface for controlling the surgical instrument; deciding a target posture of the surgical instrument corresponding to the manipulation information; deciding target state information for a driving element based on whether the target posture exceeds the driving limit for the at least one driving element provided in the surgical instrument; and driving the driving element according to the target state information.

According to an aspect, the driving element may include a joint, the target state information may include a target joint angle, and the driving limit may include a joint limit angle.

According to an aspect, the reference posture of the user input interface may be configured to be updated with posture information before a manipulation of the user input interface before a first manipulation of the user input interface.

According to an aspect, the decision of the target posture may be configured to decide the target posture based on a corresponding relationship between a predetermined movement of the user input interface and a movement of the surgical instrument.

According to an aspect, the decision of the target state information may be configured to decide modified state information in which a driving result of the driving element is constrained to a range within the driving limit as the target state information in response to a decision that the target posture exceeds the driving limit for the at least one driving element provided in the surgical instrument.

According to an aspect, the modified state information may be decided such that the driving result of the driving element approaches the driving limit as a degree to which the target posture exceeds the driving limit increases.

According to an aspect, the driving may be configured to update the reference posture of the user input interface with posture information after a manipulation of the user input interface in response to a decision that the target posture exceeds the driving limit for the at least one driving element provided in the surgical instrument.

According to an aspect, the decision of the target state information may include: deciding driving element difference information based on a degree of state change of the driving element required to change the surgical instrument to the target posture; deciding modified state information in which a driving result of the driving element is constrained to a range within the driving limit; and deciding whether the target posture exceeds a driving limit for the driving element based on the driving element difference information and the modified state information.

According to an aspect, the decision of the driving element difference information may include: deciding a current posture of the surgical instrument based on information on a current state of the driving element; deciding posture difference information based on a difference between the target posture and the current posture; and transforming the posture difference information into the driving element difference information.

According to an aspect, the decision of the modified state information may decide the modified state information based on the driving element difference information, an upward driving limit value of the driving element, a downward driving limit value of the driving element, and a tangent function.

According to an aspect, the decision of whether the target posture exceeds the driving limit may decide whether the target posture exceeds the driving limit based on whether a difference between modified driving element difference information and the driving element difference information exceeds a first predetermined threshold.

According to an aspect, the modified driving element difference information may be decided based on a difference between the modified state information and information on the current state of the driving element.

According to an aspect, the decision of the target state information may further include deciding the modified state information as the target state information in response to a decision that the driving limit is exceeded.

According to an aspect, the decision of the target state information may further include: deciding a value obtained by adding the driving element difference information to information on a current state of the driving element as the target state information in response to a decision that the driving limit is not exceeded and a decision that the driving element difference information is less than a second threshold.

According to an aspect, the decision of the target state information may be repeatedly performed until a decision is made that the driving limit is exceeded or a decision is made that the driving element difference information is less than a second threshold.

An apparatus for driving the surgical instrument according to another embodiment of the present disclosure includes: at least one processor; and at least one memory, wherein the at least one processor may be configured to: generate manipulation information based on an amount of change in a reference posture of a user input interface for controlling the surgical instrument; decide a target posture of the surgical instrument corresponding to the manipulation information; decide target state information for a driving element based on whether the target posture exceeds a driving limit for the at least one driving element provided in the surgical instrument; and drive the driving element according to the target state information.

A surgical robot system according to another embodiment of the present disclosure includes: a user input interface; a surgical instrument; and at least one processor, wherein the at least one processor may be configured to: generate manipulation information based on an amount of change in a reference posture of a user input interface for controlling the surgical instrument; decide a target posture of the surgical instrument corresponding to the manipulation information; decide target state information for a driving element based on whether the target posture exceeds a driving limit for the at least one driving element provided in the surgical instrument; and drive the driving element according to the target state information.

There is provided a computer readable storage medium having instructions executed by a processor according to another embodiment of the present disclosure, wherein the instructions allow the processor to: generate manipulation information based on an amount of change in a reference posture of a user input interface for controlling a surgical instrument; decide a target posture of the surgical instrument corresponding to the manipulation information; decide target state information for a driving element based on whether the target posture exceeds a driving limit for the at least one driving element provided in the surgical instrument; and drive the driving element according to the target state information.

In addition, another method for implementing the present disclosure, another system, and a computer-readable recording medium storing a computer program for executing the method may be further provided.

Other aspects, features, and advantages in addition to those described above will become apparent from the following drawings, claims, and detailed description of the present disclosure.

An embodiment of the present disclosure is directed to addressing an issue in which the movement of the surgical robot is interrupted by restricting the movement within the driving limit of the surgical robot and varying the driving method of the surgical robot depending on a restriction within the driving limit. When the movement is restricted within the driving limit of the surgical robot, for example, by initializing the reference posture of the user input interface of the master robot, surgery can be performed using the surgical robot by more accurately reflecting the intuitive manipulation of a user.

The benefits of the present disclosure are not limited to those mentioned above, and other benefits not mentioned may be clearly understood by those skilled in the art from the following description.

### BRIEF DESCRIPTION OF THE DRAWINGS

FIG. 1 is a diagram for explaining an example of a system for driving a surgical instrument according to an embodiment.

FIG. 2A is a configuration diagram illustrating an example of a user terminal according to an embodiment.

FIG. 2B is a configuration diagram illustrating an example of a server according to an embodiment.

FIG. 3 is a diagram for explaining another example of a surgical robot system that drives a surgical instrument according to an embodiment.

FIG. 4 is a block diagram illustrating the internal configuration of the surgical robot system of FIG. 3.

FIG. 5 is a perspective view of a slave robot of the surgical robot system of FIG. 3 and a multi-joint surgical instrument mounted thereon.

FIG. 6 is a perspective view illustrating a multi-joint type surgical instrument according to an embodiment of the present disclosure.

FIGS. 7 and 8 are perspective views of an end tool of the multi-joint type surgical instrument of FIG. 6.

FIGS. 9A to 9B is a plan view of the end tool of the multi-joint type surgical instrument of FIG. 6.

FIGS. 10 and 11 are perspective views of a driving part of the multi-joint type surgical instrument of FIG. 6

FIG. 12 is a plan view of the driving part of the multi-joint type surgical instrument of FIG. 6.

FIG. 13 is a rear view of the driving part of the multi-joint type surgical instrument of FIG. 6.

FIG. 14 is a side view of the driving part of the multi-joint type surgical instrument of FIG. 6.

FIG. 15 is a view illustrating the configuration of pulleys and wires of the multi-joint type surgical instrument illustrated in FIG. 6, in detail for the configuration related to a first jaw.

FIG. 16 is a view illustrating the configuration of pulleys and wires of the multi-joint type surgical instrument illustrated in FIG. 6, in detail for the configuration related to a second jaw.

FIGS. 17A to 18C are views illustrating a pitch motion of the multi-joint type surgical instrument illustrated in FIG. 6.

FIGS. 19A to 20B are views illustrating a yaw motion of the multi-joint type surgical instrument illustrated in FIG. 6.

FIG. 21 is a schematic flowchart of a method for driving a surgical instrument according to an aspect.

FIG. 22 is a flowchart for explaining the target state information decision stage of FIG. 21 in driving a conventional surgical instrument.

FIG. 23 exemplarily shows control results when a driving limit is reached in driving a conventional surgical instrument.

FIG. 24 shows the issue of control standard inconsistency when the driving range of a surgical robot is constrained to within the driving limit according to an aspect.

FIG. 25 is a schematic flowchart of a method for driving a surgical instrument according to an embodiment of the present disclosure.

FIG. 26 shows a conceptual information processing procedure for the target state information decision stage of FIG. 25.

FIG. 27 shows a conceptual information processing procedure of a reference posture initialization procedure when the driving limit of FIG. 25 is reached.

FIG. 28 is an exemplary detailed flowchart of the target state information decision stage of FIG. 26 according to an embodiment of the present disclosure.

FIG. 29 is an exemplary detailed flowchart of the driving element difference information decision stage of FIG. 28.

### DETAILED DESCRIPTION

Hereinafter, various embodiments of the present disclosure are described in conjunction with the accompanying drawings. Various embodiments of the present disclosure may make various changes and have various embodiments, and specific embodiments are illustrated in the drawings and related detailed descriptions are described. However, this is not intended to limit the various embodiments of the present disclosure to specific embodiments, and should be understood to include all changes and/or equivalents or substitutes included in the spirit and technical scope of the various embodiments of the present disclosure. In connection with the description of the drawings, similar reference numerals have been used for similar components.

Expressions such as “comprise” or “may comprise” that may be used in various embodiments of the present disclosure indicate the presence of the corresponding function, operation, or component disclosed, and do not limit one or more additional functions, operations, or components. In addition, in various embodiments of the present disclosure, terms such as “comprise” or “have” are used to specify the presence of stated features, integers, steps, operations, components, parts, or a combination thereof, but do not preclude the presence or addition of one or more other features, integers, steps, operations, components, parts, or combinations thereof.

In various embodiments of the present disclosure, the expression such as “or” includes any and all combinations of words listed together. For example, “A or B” may include A, B, or both A and B.

Although the expressions such as “first,” “second,” etc. used in various embodiments of the present disclosure may modify various components of the various embodiments, but do not limit the components. For example, the expressions do not limit the order and/or importance of corresponding components. These expressions may be used to distinguish one component from the other components. For example, a first user device and a second user device are both user devices and represent different user devices. For example, a first component may be referred to as a second component without departing from the scope of right of various embodiments of the present disclosure, and similarly, the second component may also be referred to as the first component.

In an embodiment of the present disclosure, terms such as “module,” “unit,” or “part” are used to refer to components that perform at least one function or operation, and these components may be implemented as hardware or software, or as a combination of hardware and software. In addition, a plurality of “modules,” “units,” “parts,” etc. may be integrated into at least one module or chip and implemented with at least one processor, except in the cases where each thereof needs to be implemented with individual specific hardware.

Terms used in various embodiments of the present disclosure are merely used to describe specific embodiments and are not intended to limit the various embodiments of the present disclosure. A singular expression includes a plural expression, unless the context clearly states otherwise.

Unless otherwise defined, all terms including technical and scientific terms used herein have the same meaning as commonly understood by those having ordinary skill in the art to which various embodiments of the present disclosure pertains.

It will be further understood that terms, such as those defined in commonly used dictionaries, should be interpreted as having a meaning that is consistent with their meaning in the context of the relevant art and will not be interpreted in an idealized or overly formal sense unless expressly so defined in various embodiments of the present disclosure.

Hereinafter, various embodiments of the present disclosure will be described in detail using the accompanying drawings.

Laparoscopic surgery refers to a surgery performed by forming a hole in the abdominal cavity of a patient, inserting a narrow and long tube through the hole, and using surgical instruments connected to the end. The surgical instrument may be, for example, an articulated instrument.

In this connection, when a passive surgical instrument is used, the surgical instrument and a control unit operated by a user move symmetrically with respect to a hole in the abdominal cavity, so that more than a certain period of practice is needed until a user becomes familiar with the control. In addition, since the surgical instruments may not be checked with the naked eye, the surgical instruments need to be manipulated while a surgical operator watches the camera images acquired by inserting an endoscopic camera into the abdominal cavity.

This situation is the same even when laparoscopic surgery is performed using a surgical robot system, but there is a benefit of being intuitively controlled compared to manual surgical instruments. As will be described later in the description, the surgical robot system according to an embodiment includes a master robot and a slave robot. The slave robot may be referred to as a surgical robot or surgical instrument, and may refer to a configuration that performs surgery by acting directly on a patient. The master robot may be referred to as a master device or a user input interface, and may refer to a configuration for receiving a user manipulation to control the slave robot.

This type of surgical robot system is mounted with articulated instruments and separates the portion that performs surgery (for example, surgical robot) and the portion that a user manipulates (for example, the master device), and thus intuitive control is possible compared to manual surgical instruments. In other words, the surgical robot system is capable of controlling operations so that surgical instruments may be intuitively controlled by matching the movements of a user with the movements on the laparoscopic camera screen.

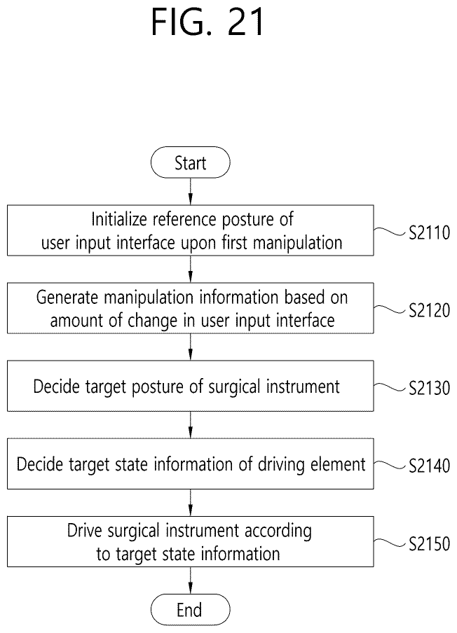

FIG. 21 is a schematic flowchart of a method for driving a surgical instrument according to an aspect. With reference to FIG. 21, the operation process of the surgical robot system, that is, the process of manipulating the surgical robot mounted with surgical instruments through the master device, will be described in more detail.

First, as illustrated in FIG. 21, when a user starts controlling the surgical robot, the reference posture information of the master device is initialized (stage 2110). As a non-limiting example, prior to the first manipulation of the user input interface provided in the master device, the reference posture of the user input interface may be initialized to the current posture of the user input interface. According to an aspect, control of the surgical robot may be performed based on the degree to which the user input interface has changed, so the reference posture of the user input interface, which serves as a reference for determining the degree of change, is initialized prior to manipulation by a user.

Thereafter, a user manipulates the master device (for example, the user input interface), and the master device generates manipulation information based on the reference posture information. For example, the master device may generate manipulation information based on an amount of change from the reference posture of the user input interface (stage 2120). The generated manipulation information may be transmitted to the surgical robot.

The surgical robot generates a target posture through the received manipulation information (stage 2130). For example, the target posture of the surgical instrument corresponding to the manipulation information may be decided. The surgical robot goes through a process of generating a target joint angle to create the target posture (stage 2140). For example, the surgical robot may decide target state information of at least one driving element. Herein, as a non-limiting example, the driving element may be a joint provided in the surgical robot, and the target state information may be the target joint angle. When the target state information, for example, the target joint angle, is successfully generated, the surgical robot performs driving with the target joint angle (stage 2150).

A joint, which is an exemplary driving element, refers to a mechanical structure that is mounted with an actual motor and may rotate, and the joint movement of the surgical robot includes the movement of both a robot arm and the articulated instrument mounted on the surgical robot. In addition, the process of receiving the target posture of the surgical robot as an input and generating the target joint angle of the surgical robot is defined as “inverse kinematics transformation.” The inverse kinematics transformation is an algorithm decided by the target posture, mechanical structure of a robot, and angle constraints of each joint, and at least one algorithm among various algorithms may be adopted for inverse kinematics transformation. In this description, the Newton-Raphson numerical inverse kinematics, which is widely known as a numerical analysis method among various algorithms for inverse kinematics transformation, may be used as a reference. However, this is merely an example for convenience of explanation, and the technical idea of the present disclosure is not limited thereto.

Numerical analysis inverse kinematics transformation has the benefit of being able to implement a universal algorithm that is not limited by the mechanical characteristics of a robot. In other words, there is no need to modify the algorithm itself, through a structure that utilizes the information without relying thereon even when the mechanical characteristics of the robot change. However, such being the case, even when a target posture that may not generate the target joint angle is input due to the mechanical structure or joint angle constraints of the robot, it is not possible to determine whether the target posture is feasible. Accordingly, most numerical analysis inverse kinematics transformation algorithms determine that the inverse kinematics transformation has failed when the target joint posture is not output even after a certain amount of time has elapsed from the start of calculation.

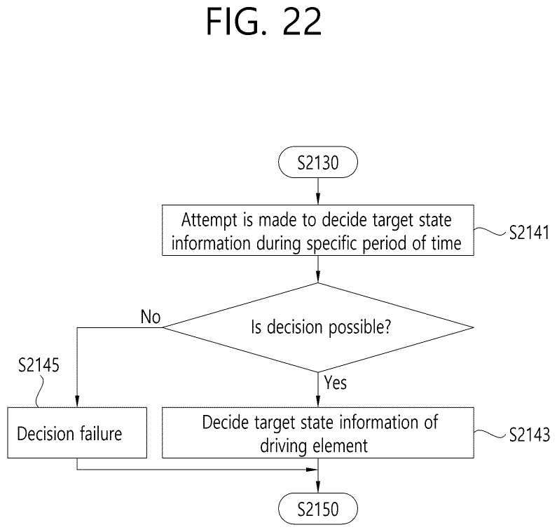

FIG. 22 is a flowchart for explaining the target state information decision stage of FIG. 21 in driving a conventional surgical instrument. As illustrated in FIG. 22, when the target state information is decided based on a conventional numerical analysis inverse kinematics transformation, an attempt is made to decide the target state information during a time interval of a specific predetermined length (stage 2141). For example, an attempt may be made to decide a target joint angle to achieve the target posture corresponding to the manipulation information of the user input interface. When the target state information is able to be decided, the target state information for achieving the target posture is decided (stage 2143). However, when the target state information is unable to be decided during a predetermined time period, the target state information is not decided, decision failure (stage 2145) is finalized, and the process ends.

Such transform failure in the existing numerical analysis inverse kinematics transformation greatly hinders user manipulability during manipulation of the surgical robot. When a target posture that the surgical robot is unable to implement due to limitations in mechanical structure or joint angles is input, the surgical robot will stop driving for a certain period of time, inevitably interrupting the surgical operation. In other words, even though the manipulation information of the user input interface is changed, the corresponding target state information is not decided so the driving posture of the surgical robot does not change, resulting in interruptions in the surgical operation.

In addition, the transform failure itself also greatly hinders user manipulability. In this regard, FIG. 23 exemplarily shows control results 2310 for the surgical instrument when a driving limit is reached in driving a conventional surgical instrument.

The target posture of the surgical robot changes because the posture information of the master device manipulated by a user changes, which is independent of whether the inverse kinematics transformation fails. Accordingly, when the inverse kinematics transformation fails, the surgical robot maintains the target posture that was successfully transformed to the previous inverse kinematics, not the latest one. Thereafter, when a new target posture capable of inverse kinematics transformation is received from the master device, there is a high possibility that the robot will move suddenly due to a difference in posture. The user will perceive the situation as an inconvenience in which the robot suddenly moves significantly, regardless of the manipulation intention.

For example, as illustrated in FIG. 23, a first manipulation 2311 of the user input interface by a user may display the corresponding operation of the surgical instrument through a camera screen 2321. Herein, when the posture or position in which the surgical instrument is to be driven according to the first manipulation corresponds to an inverse kinematics transformation failure posture area 2330, the surgical instrument is not driven despite the first manipulation 2311 and maintains the target posture previously successfully transformed to inverse kinematics.

Thereafter, the corresponding operation of the surgical instrument when a second manipulation 2313 of the user input interface by a user is performed may be displayed through a camera screen 2323. When the target state of the surgical instrument is set to a state in which inverse kinematics transformation is possible rather than the inverse kinematics transformation failure posture area 2330 according to the first manipulation 2311 and the second manipulation 2313, the surgical instrument may be rapidly driven 2333 from an inverse kinematics transformation successful posture before the first manipulation 2311 to the posture according to the second manipulation 2313. Accordingly, the user intended the movement to lead to the first manipulation 2311 and the second manipulation 2313. However, contrary to the intention of the user, no driving occurs on the surgical instrument at the time of the first manipulation 2311, but a sudden large movement occurs in response to the second manipulation 2313, causing discomfort.

To address this issue, for example, rather than failing the inverse kinematics transformation, it may be considered to allow the inverse kinematics transformation to be completed by satisfying the given characteristics of the surgical instrument even when it is different from the input target posture. Herein, the characteristics that may be given may include, for example, driving limits such as the joint limit angles of a robot. When a specific joint angle is outside the limit value during the inverse kinematics transformation, the transform may be performed while maintaining the joint angle at the limit value to output implementable joint information. As in the typical numerical analysis inverse kinematics transformation procedure, when the joint angle is outside the limit value during the transform, the transform itself is not retried from the beginning, but the transform may be continued while maintaining the angle within the limit value.

However, when only the inverse kinematics transformation method that involves constraints within the driving limit is applied, other usability-impairing issues may arise.

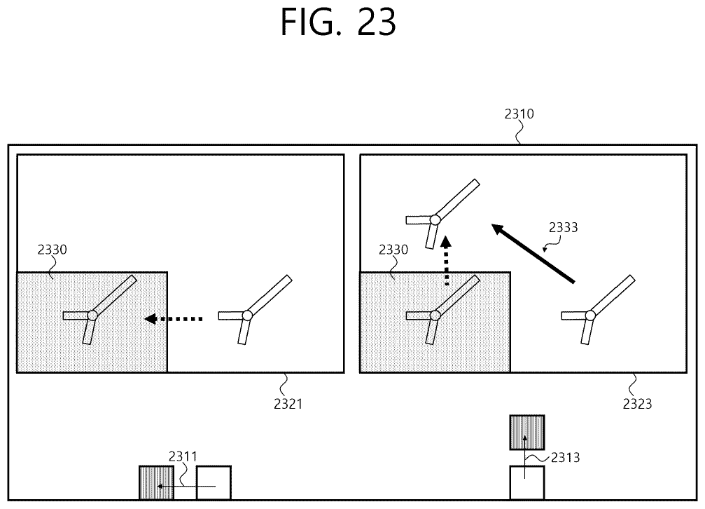

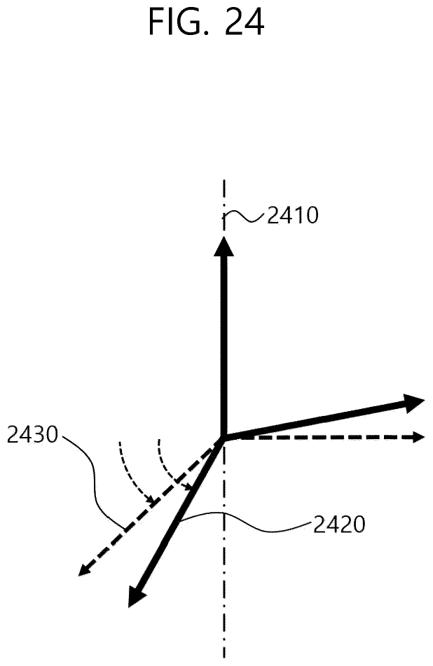

FIG. 24 shows the issue of control standard inconsistency when the driving range of a surgical robot is constrained to within the driving limit according to an aspect. For example, in the case where the driving element is a joint, when the angle of a specific joint is constrained to a limit value, the posture of the surgical robot created with the corresponding joint angles differs from the target posture received from the master device. As illustrated in FIG. 24, a target posture 2420 input by a user and the master device based on a specific axis 2410 on the coordinate system that caused a joint limit constraint may exceed a joint limit and be controlled to a constrained posture 2430 within the joint limit. In other words, according to the manipulation of the user through the user input interface of the master device, it needs to be driven to the target posture 2420, but due to the joint limitations of joints operating along the specific axis 2410, it may be considered to control the joints to operate only up to a modified posture 2430 by constraining the joint limitations from being exceeded. However, when the target posture input by the user and the master device is different from the posture in which the surgical robot or surgical instrument is actually driven, the coordinate system including a rotation axis, which is the standard for inputting direction information, also changes. In other words, after the first manipulation within the joint limit is made, when the user subsequently wishes to control the surgical robot through the user input interface, the user intends to perform subsequent control based on the current posture of the surgical robot, that is, the posture 2430 driven within a limited range, by checking the posture of the surgical robot secured through a camera, for example. However, the user input interface previously manipulated by the user exists in a state corresponding to the surgical robot being driven to the target posture 2420, which creates a direction change that is different from the intuitive expectation of the user when the user changes the direction of the surgical robot through the master device.

To address this issue, it is possible to consider directly modifying the target posture input to the inverse kinematics transformation based on the posture created as a result of the joint limit angle constraint algorithm. However, the method of directly modifying posture information or direction information numerically may not avoid the issue of accumulating numerical errors based on computer calculations, and as a result, it has a limitation in that the modified target posture may cause issues such as unexpected sudden changes in robot joint angles.

The method and apparatus for driving the surgical instrument according to an embodiment of the present disclosure enable inverse kinematics transformation even when some or all of the joints of the surgical robot reach a limit angle and simultaneously do not hinder usability when a user controls the surgical robot by manipulating the user input interface of the master device. The surgical robot may perform the aforementioned joint limit constraint algorithm. When inverse kinematics transformation is performed with one or more joints reaching the limit value, the reference posture information of the surgical robot and the master device may be initialized. When the reference posture information of the surgical robot and the master device is initialized, the posture of the surgical robot expressed by the constrained joints and the target posture input by the master device become the same, addressing the issue of differences in coordinate systems including the rotation axis. Herein, the user may feel the driving of the surgical robot stop due to communication delay in the process of receiving signals from the surgical robot and initializing the reference posture information of the master device. However, for example, by using communication technologies such as EtherCAT communication or TCP communication, the communication delay may be implemented to be significantly shorter than the reference time for determining the failure of inverse kinematics transformation.

Hereinafter, a method and apparatus for driving a surgical instrument according to embodiments of the present disclosure will be described in more detail with reference to the drawings.

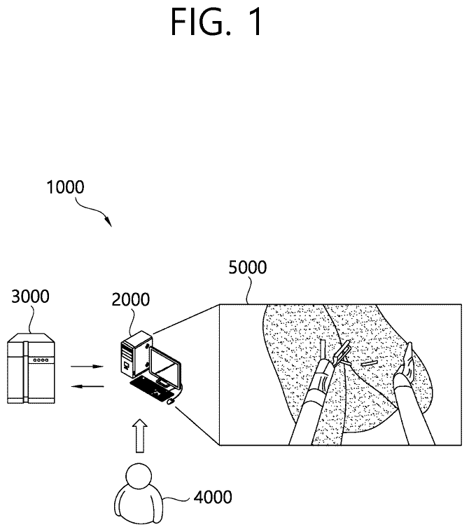

FIG. 1 is a diagram for explaining an example of a system for driving a surgical instrument according to an embodiment.

Referring to FIG. 1, a system 1000 includes a user terminal 2000 and a server 3000. For example, the user terminal 2000 and the server 3000 may be connected to each other through a wired or wireless communication method to transmit and/or receive data to and/or from each other.

For convenience of explanation, although FIG. 1 illustrates that the system 1000 includes the user terminal 2000 and the server 3000, an embodiment of the present disclosure is not limited thereto. For example, other external devices (not shown) may be included in the system 1000, and operations of the user terminal 2000 and the server 3000 to be described below may be implemented by a single device (for example, the user terminal 2000 or the server 3000) or a plurality of devices.

The user terminal 2000 may be a computing apparatus that is provided with a display apparatus and a device (for example, a keyboard, a mouse, or the like) for receiving a user input, and includes a memory and a processor. For example, the display apparatus may be implemented as a touch screen to receive user input. For example, the user terminal 2000 may correspond to a notebook PC, a desktop PC, a laptop, a tablet computer, a smartphone, or the like, but is not limited thereto.

The server 3000 may be an apparatus that communicates with an external device (not shown) including the user terminal 2000. As an example, the server 3000 may be an apparatus that stores various types of data.

Alternatively, the server 3000 may be a computing apparatus including a memory and a processor, and having its own computing capability. For example, the server 3000 may perform at least some of operations of the user terminal 2000 to be described below with reference to the drawings. For example, the server 3000 may also be a cloud server, but is not limited thereto.

According to an aspect, the user terminal 2000 may drive the surgical instrument. In this description, the method for driving the surgical instrument below may be described as being performed by a computing device. The computing device may be, for example, the user terminal 2000 or the server 3000, but is not limited thereto. Any single or plural computing devices including a processor may configure a computing device. Hereinafter, for convenience of explanation, the control procedure of the surgical instrument by the user terminal 2000 may be described, but this is only for explanation, and the method of controlling the surgical instrument according to embodiments of the present disclosure may be performed by any computing device.

The user terminal 2000 may generate manipulation information, which refers to information representing the operations of a user for driving the surgical instrument. For example, the manipulation information may include position information and/or direction information on a three-dimensional coordinate system capable of manipulating the position and function of the surgical instrument by the operations of the user. In this description, the manipulation information may be decided based on an amount of change in the reference posture of the user input interface, such as a manipulation lever, for example.

The target posture of the surgical instrument may refer to a posture expected to be assumed as a result of the surgical instrument operating in response to manipulation information. The manipulation information, in other words, the movement of the surgical instrument corresponding to the degree to which the user input interface has changed, may be decided by a predetermined correspondence relationship, and this correspondence relationship may vary depending on the embodiment or setting. For example, for more detailed control, the movement of the surgical instrument corresponding to an amount of change in the user input interface may be set to be smaller, and for more immediate and rapid control, the movement of the surgical instrument corresponding to the amount of change in the user input interface may be set to be larger. According to these settings, the target posture of the surgical instrument corresponding to the manipulation information for the user input interface may be decided.

The target state information for the driving element includes a state value that at least one driving element of the surgical instrument needs to take in order for the surgical instrument to reach the target posture. For example, in an embodiment of the present disclosure, the driving element of the surgical instrument may include at least one joint, and target state information according to an aspect may include the target joint angle, without being limited thereto. For example, inverse kinematics transformation may be performed to obtain the target joint angle for at least one joint of the surgical instrument based on the target posture of the surgical instrument. According to an aspect, the inverse kinematics transformation may follow Newton-Raphson numerical inverse kinematics based on numerical analysis, without being limited thereto. For example, any of the various inverse kinematics transformation methods that exist or will be developed later, such as inverse kinematics transformation using an artificial neural network, may be applied. Hereinafter, for convenience of explanation, it may be described based on Newton-Raphson numerical inverse kinematics.

According to an aspect of the present disclosure, the user terminal 2000 may decide target state information for the driving element based on whether the target posture of the surgical instrument corresponding to the manipulation information exceeds a driving limit for the at least one driving element of the surgical instrument. According to an aspect, the target state information may be the target joint angle, but is not limited thereto. For example, when the target joint angle corresponding to the target posture is within the joint limit angle, the target joint angle is repeatedly calculated according to a typical numerical analysis method until a preset threshold is satisfied. In addition, when the difference between the target joint angle and the current angle is sufficiently smaller than the threshold, the target joint angle may be finalized. On the other hand, when the target joint angle corresponding to the target posture is outside the joint limit angle, a modified safe joint angle may be decided by limiting the target joint angle to within the joint limit angle, and this safe joint angle may be finalized as the final target joint angle.

Thereafter, the driving elements of the surgical instrument, for example a joint, may be driven based on the finalized target state information. According to an aspect, when the modified target state information constrained within the driving limit for the at least one driving element is applied, the reference posture of the user input interface may be initialized to the state after manipulation by a user. Accordingly, an embodiment of the present disclosure is configured to remove obstacles to intuitive control that arise from differences between the reference posture recognized by the user from a current state of the surgical instrument and the reference posture of the user input interface, and to enable the driving of the surgical instrument more reliably according to the intuitive intention of the user.

Herein, the application of FIG. 1 may be a software program installed for the purpose of driving the surgical instrument of a user 4000. For example, through the application, the user 4000 may perform various activities such as generating manipulation information based on an amount of change in the reference posture of the user input interface for controlling the surgical instrument, deciding the target posture of the surgical instrument corresponding to the manipulation information, deciding the target state information for the driving element based on whether the target posture exceeds the driving limit for the at least one driving element provided in the surgical instrument, or driving the driving element according to the target state information.

The user terminal 2000 may output an image 5000 representing the operation of the surgical instrument driven based on the operation of the user 4000. For example, the user terminal 2000 may generate manipulation information based on an amount of change in the reference posture of the user input interface for the user 4000 to control the surgical instrument. Then, the user terminal 2000 may decide the target posture of the surgical instrument corresponding to the manipulation information, and decide the target state information for the driving element based on whether the target posture exceeds the driving limit for the at least one driving element provided in the surgical instrument. Subsequently, the user terminal 2000 may drive the driving element according to the decided target state information and output the image 5000 representing the operation of the surgical instrument driven in this way. The user 4000 may intuitively understand the operation of the surgical instrument according to the operation of the user through the image 5000 representing the operation of the surgical instrument and manipulate the surgical instrument more accurately.

As described above, at least some of the operations of the user terminal 2000 described below with reference to the drawings may be performed by the server 3000. For example, the server 3000 may perform various activities such as generating manipulation information based on an amount of change in the reference posture of the user input interface for controlling the surgical instrument, deciding the target posture of the surgical instrument corresponding to the manipulation information, deciding the target state information for the driving element based on whether the target posture exceeds the driving limit for the at least one driving element provided in the surgical instrument, or driving the driving element according to the target state information. Alternatively, at least some of these activities may be performed by the server 3000, and at least some thereof may be performed by the user terminal 2000.

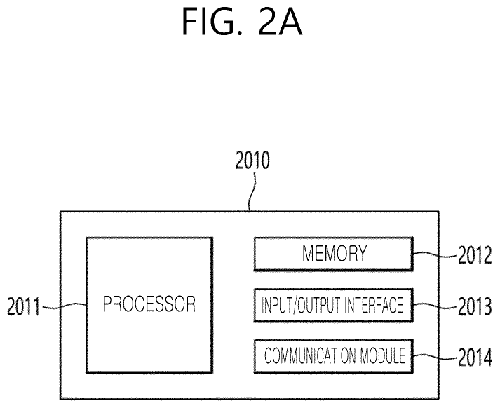

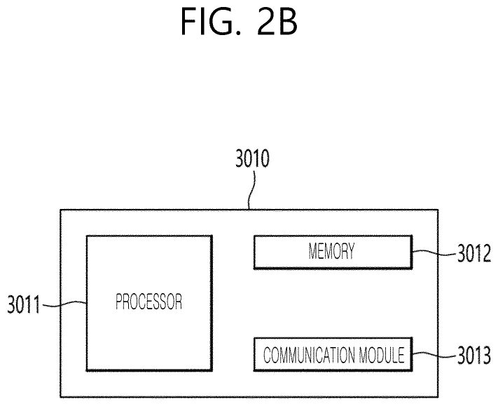

FIG. 2A is a configuration diagram illustrating an example of a user terminal according to an embodiment.

Referring to FIG. 2A, a user terminal 2010 includes a processor 2011, a memory 2012, an input/output interface 2013, and a communication module 2014. For convenience of explanation, FIG. 2A illustrates only components related to an embodiment of the present disclosure. Accordingly, the user terminal 2010 may further include other general-purpose components, in addition to the components illustrated in FIG. 2A. In addition, it is obvious to those skilled in the technical field to which the present disclosure pertains that the processor 2011, the memory 2012, the input/output interface 2013, and the communication module 2014 illustrated in FIG. 2A may also be implemented as independent devices.

The processor 2011 may process commands of a computer program by performing basic arithmetic, logic, and input/output operations. Herein, the commands may be provided from the memory 2012 or an external device (for example, the server 3000, etc.). In addition, the processor 2011 may control the overall operation of other components included in the user terminal 2010.

First, the processor 2011 generates manipulation information regarding the operations of a user to drive the surgical instrument. For example, the processor 2011 may generate manipulation information regarding the operation of the user based on a member that allows the position and function of the surgical instrument to be manipulated by the operation of the user.

The member for manipulating the position and function of the surgical instrument by the operation of a user may be formed in the form of a handle-shaped manipulation member, but is not limited thereto and may be modified and implemented in various shapes to achieve the same purpose. For example, some may be formed in the shape of a handle, and the others may be formed in a different shape, such as a clutch button. In addition, a finger insertion tube may be formed so as to allow the finger(s) of a surgical operator to be inserted therethrough and fixed to facilitate manipulation of a surgical tool. Hereinafter, in this description, a member that allows manipulation by the operation of the user may also be referred to as the user input interface.

Herein, before the first manipulation of a user of the user input interface, the processor 2011 may update the reference posture of the user input interface with the posture information before manipulation of the user input interface. Since the driving of the surgical instrument by the user may be performed based on the degree to which the user input interface has changed by the user. Hence, by initializing the reference posture of the user input interface to the state before the manipulation before the user performs the first manipulation, the difference between the state of the user input interface after user manipulation and the state of the user input interface before user manipulation, in other words, an amount of change in the user input interface, may be decided.

The processor 2011 may generate manipulation information based on an amount of change in the reference posture of the user input interface. The manipulation information refers to information representing the intuitive operation of a user to manipulate the position and function of the surgical instrument. More specifically, but non-limitingly, the manipulation information may include position information and orientation information on a physical coordinate system of a member that allows a user to manipulate the position and function of the surgical instrument. As an example, the manipulation information may include a transformation matrix representing linear and rotational movement in a homogeneous coordinate system. The transformation matrix may be a homogeneous transformation matrix and may include rotation matrix information and translation vector information. As another example, the manipulation information may include position information and orientation information on a physical coordinate system expressed according to an expression method such as a screw. However, the examples of manipulation information are not limited to the above. The manipulation information may be decided based on an amount of change in the reference posture of the user input interface. Herein, the manipulation information may represent an amount of change with respect to the reference posture, and the reference posture may represent the degree of change of the user input interface with respect to the origin. However, the reference posture and manipulation information may be expressed, for example, by a homogeneous transformation matrix or a screw method as described above.

The processor 2011 may generate manipulation information based on a member that allows a user to manipulate the position and function of the surgical instrument, for example, position information and orientation information of the user input interface. For example, the processor 2011 may generate manipulation information using the difference between the initial position information and initial orientation information of the member that allows the user to manipulate the position and function of the surgical instrument, and the position information and orientation information after the operation of the user of the aforementioned member. According to an aspect, the processor 2011 may generate manipulation information based on an amount of change from the reference posture of the user input interface according to the manipulation of the user.

In addition, based on the manipulation information, the processor 2011 may decide the target posture of the surgical instrument corresponding to the manipulation information. For example, the processor 2011 may decide the target posture of the multi-joint surgical instrument based on the manipulation information. According to an aspect, the processor 2011 may be configured to decide the target posture based on the correspondence relationship between a predetermined movement of the user input interface and the movement of the surgical instrument.

The processor 2011 decides the target state information for the at least one driving element provided in the surgical instrument, considering whether the target posture exceeds the driving limit for the at least one driving element provided in the surgical instrument. As a non-limiting example, at least one driving element of the surgical instrument may include a joint, and the target state information of the surgical instrument may include a target joint angle. Additionally, the driving limits for the driving element may include joint limit angles.

For example, processor 2011 may be configured to decide the modified state information in which the driving result of the driving element is constrained to a range within the driving limit as the target state information in response to a decision that the target posture exceeds a driving limit for the at least one driving element provided in the surgical instrument. In other words, as a non-limiting example, when it is determined that at least one joint included in the surgical instrument needs to exceed the joint limit angle according to the target posture, the processor 2011 may decide target state information, in other words, target joint angle, to have a joint angle limited to a range within the joint limit angle.

Herein, the modified state information may be decided so that the driving result of the driving element approaches the driving limit as the degree to which the target posture exceeds the driving limit increases. For example, when the joint angle according to the target posture exceeds the joint limit angle by 10 degrees, a modified target joint angle may be decided to have a value closer to the joint limit angle compared to a case where the joint angle according to the target posture exceeds the joint limit angle by 5 degrees. As a non-limiting example, assuming that the joint limit angle is 90 degrees, when the target joint angle according to the target posture is 100 degrees, the modified target joint angle of 89.95 degrees may be decided. In addition, when the target joint angle according to the target posture is 95 degrees, the modified target joint angle of 89.90 degrees may be decided.

For example, the processor 2011 may decide driving element difference information based on the degree of change in the state of the driving element required to change the surgical instrument to the target posture, decide modified state information by which the driving result of the driving element is constrained to a range within the driving limit, and decide whether the target posture exceeds a driving limit for the driving element based on the driving element difference information and the modified state information.

In other words, the processor 2011 first decides the driving element difference information by calculating the extent to which the state of the driving element needs to be changed to reach the target posture, regardless of whether the driving limit is reached. According to an aspect, the processor 2011 may first decide the current posture of the surgical instrument based on information on the current state of the driving element, and then decide posture difference information based on the difference between the target posture and the current posture. The driving element difference information may be decided by transforming the decided posture difference information in this way into the driving element difference information using a physical quantity representing the kinematic information of a robot, such as a Jacobian matrix.

In addition, the processor 2011 may decide the modified state information, which is a value that constrains the operating range of the driving element to within the driving limit, in the case in which the operating range of the driving element exceeds the driving limit. According to an aspect, the processor 2011 may decide the modified state information using a tangent function based on previously decided driving element difference information and the upward driving limit value of the driving element and the downward driving limit value of the driving element. Non-limiting but more specific mechanisms for deciding the modified state information are described in detail later in this description. For example, when the driving element is a joint, safe joint information, which is a joint angle constrained within a joint limit, is described as an example of the modified state information.

Subsequently, the processor 2011 may decide whether the target posture of the surgical instrument corresponding to the manipulation information exceeds the driving limit for the at least one driving element provided in the surgical instrument by comparing the decided driving element difference information and the modified state information. For example, the processor 2011 may decide whether the driving limit is exceeded based on whether the difference between the modified driving element difference information and the driving element difference information exceeds a first predetermined threshold. Herein, the modified driving element difference information may represent the difference between the modified state information and information on the current state of the driving element. In other words, the processor 2011 may calculate a difference value between driving element difference information decided without constraints on the driving limit and modified driving element difference information decided to be constrained within the driving limit when the driving limit is reached. When this difference exceeds a first predetermined threshold, it may be decided that the driving limit has been exceeded and needs to be constrained to within the driving limit. As a non-limiting but more specific example, the first threshold may be a difference determination constant, as described in detail later in this description.

When it is decided whether the driving limit for the at least one driving element is exceeded, the processor 2011 may decide target state information for the driving element based on whether the driving limit is exceeded. For example, processor 2011 may decide the modified state information as the target state information in response to a decision that a driving limit is exceeded. In other words, when the target posture of the surgical instrument corresponding to the manipulation information exceeds the driving limit of the at least one driving element provided in the surgical instrument, the processor 2011 may decide the modified state information, which is a value constrained within the driving limit, as the target state information for the driving element. Accordingly, according to an embodiment of the present disclosure, when the inverse kinematics transformation is performed according to a conventional numerical analysis method, a determination of failure of inverse kinematics transformation is performed only after a predetermined time has elapsed due to exceeding the driving limit, thereby addressing the issue of the surgical operation of the surgical instrument being interrupted due to a delay in the algorithm calculation time.

In response to a decision that the driving limit is not exceeded and the decision that the driving element difference information is less than a second threshold, the processor 2011 may decide a value of adding the driving element difference information to the information on the current state of the driving element as the target state information. The processor 2011 may repeatedly perform the procedure of deciding the target state information until a decision is made that the driving limit is exceeded or a decision is made that the driving element difference information is less than the second threshold. In other words, the processor 2011 may decide that the operating limit has been exceeded, or repeatedly perform a decision of the driving element difference information until deciding that the driving element difference information is less than a second threshold, a decision of the modified state information, a decision of whether the driving limit is exceeded, and a decision of whether the driving element difference information is less than a second threshold. Herein, the second threshold is a non-limiting example and may be an epsilon determination constant, and may be represented by a reference value for determining a value sufficiently close to 0 during computer calculations. When the driving limit is not exceeded, the processor 2011 repeatedly performs the procedure until the driving element difference information becomes smaller than the epsilon determination constant to calculate driving element difference information to achieve a target posture corresponding to the manipulation information of a user, and add the calculated driving element difference information to information on the current state of the surgical instrument to finalize the target state information.

Once the target state information is decided, the processor 2011 may drive the at least one driving element provided in the surgical instrument according to the target state information. For example, the processor 2011 may control the joint to have the target joint angle by driving at least one joint provided in the surgical instrument according to the decided target joint angle.

The processor 2011 may be configured to update the reference posture of the user input interface with posture information after manipulating the user input interface in response to a decision that the target posture exceeds the driving limit for the at least one driving element provided in the surgical instrument. As seen above, when the driving elements of the surgical instrument are driven in a limited state within the driving limit, a user sees the state of the surgical instrument in a limited state and performs subsequent control based thereon. However, the movement of the user input interface is operated based on the actual state manipulated by the user rather than a restricted state, and thus a difference occurs between the reference state of the control recognized by the user and the reference state of the user input interface recognized by the processor 2011. Hence, control may be performed somewhat differently from a user intention, which may be a factor hindering intuitive control. Accordingly, according to an embodiment of the present disclosure, when the driving range of the driving element is limited to within the driving limit, the reference posture of the user input interface is updated to the state after manipulation by the user of the user input interface. In addition, by matching the state that may be recognized through the current state of the user input interface and the current operating state of the surgical instrument, the user may perform intuitive control through the user input interface.

Specific examples in which the processor 2011 according to an embodiment operates will be described in detail later in this description with reference to the drawings.

The processor 2011 may be implemented as an array of a plurality of logic gates, or may be implemented as a combination of a general-purpose microprocessor and a memory in which a program executable in the microprocessor is stored. For example, the processor 2011 may include a general-purpose processor, a central processing unit (CPU), a microprocessor, a digital signal processor (DSP), a controller, a microcontroller, a state machine, and the like. In some circumstances, the processor 110 may include an application-specific integrated circuit (ASIC), a programmable logic device (PLD), a field programmable gate array (FPGA), or the like. For example, the processor 2011 may refer to a combination of processing devices, such as a combination of a digital signal processor (DSP) and a microprocessor, a combination of a plurality of microprocessors, a combination of one or more microprocessors coupled with a digital signal processor (DSP) core, or a combination of any other configurations.

The memory 2012 may include any non-transitory computer-readable recording medium. In an embodiment, the memory 2012 may include a permanent mass storage device such as a random access memory (RAM), a read only memory (ROM), a disk drive, a solid state drive (SSD), a flash memory, etc. In another embodiment, a permanent mass storage device such as a ROM, SSD, a flash memory, a disk drive, etc. may be a separate permanent storage device which is distinguishable from the memory. In addition, an operating system (OS) and at least one program code (for example, a code for the processor 2011 to perform an operation to be described later with reference to the drawings) may be stored in the memory 2012.

These software components may be loaded from a computer-readable recording medium separate from the memory 2012. The separate computer-readable recording medium may be a recording medium that may be directly connected to the user terminal 2010, for example, a computer-readable recording medium, such as a floppy drive, a disk, a tape, a DVD/CD-ROM drive, a memory card, or the like. In addition, the software components may be loaded into the memory 2012 through the communication module 2014 instead of a computer-readable recording medium. For example, at least one program may be loaded into the memory 2012 based on a computer program (for example, a computer program for performing, by the processor 2011, an operation to be described later with reference to the drawings) installed by the files provided through the communication module 140 by developers or a computer file distribution system that distributes the installation files of applications. The input/output interface 2013 may be a member for an interface with a device (for example, a keyboard, a mouse, etc.) for input or output, the member being connected to the user terminal 2010 or being included in the user terminal 2010. The input/output interface 2013 may be configured separately from the processor 2011, without being limited thereto, and the input/output interface 2013 may be configured to be included in the processor 2011.

The communication module 2014 may provide a configuration or function for the server 3000 and the user terminal 2010 to communicate with each other through a network. In addition, the communication module 2014 may provide a configuration or function for the user terminal 2010 to communicate with another external device. For example, a control signal, a command, data, etc. provided according to the control of the processor 2011, may be transmitted to the server 3000 and/or an external device through the communication module 2014 and the network.

Although not illustrated in FIG. 2A, the user terminal 2010 may further include a display apparatus. For example, the display apparatus may be implemented as a touch screen. Alternatively, the user terminal 2010 may be connected to an independent display apparatus through a wired or wireless communication method to transmit and/or receive data to and/or from each other. For example, a video or image of driving the surgical instrument using driving information may be provided through the display apparatus.

FIG. 2B is a configuration diagram illustrating an example of a server according to an embodiment.

Referring to FIG. 2B, the server 3010 includes a processor 3011, a memory 3012, and a communication module 3013. For convenience of explanation, FIG. 2B illustrates only components related to an embodiment of the present disclosure. Accordingly, the server 3010 may further include other general-purpose components, in addition to the components illustrated in FIG. 2B. In addition, it is obvious to those skilled in the technical field to which the present disclosure pertains that the processor 3011, the memory 3012, and the communication module 3013 illustrated in FIG. 2B may also be implemented as independent devices.

The processor 3011 may perform various activities such as generating manipulation information based on an amount of change in the reference posture of the user input interface for controlling the surgical instrument, deciding the target posture of the surgical instrument corresponding to the manipulation information, deciding the target state information for the driving element based on whether the target posture exceeds the driving limit for the at least one driving element provided in the surgical instrument, or driving the driving element according to the target state information.

In other words, at least one of the operations of the processor 2011 described above with reference to FIG. 2A may be performed by the processor 3011. In this connection, the user terminal 2010 may output information transmitted from the server 3010 through the display apparatus.

Since the implementation example of the processor 3011 is the same as the implementation example of the processor 2011 described above with reference to FIG. 2A, the detailed description thereof is omitted.

The memory 3012 may store various pieces of data, such as data necessary for the operation of the processor 3011 and data generated according to the operation of the processor 3011. Additionally, an operating system (OS) and at least one program (for example, a program necessary for the processor 3011 to operate, etc.) may be stored in the memory 3012.

Since the implementation example of the memory 3012 is the same as the implementation example of the memory 2012 described above with reference to FIG. 2A, the detailed description thereof will be omitted.

The communication module 3013 may provide a configuration or function for the server 3010 and the user terminal 2010 to communicate with each other through a network. Additionally, the communication module 2014 may provide a configuration or function for the server 3010 to communicate with other external devices. For example, control signals, commands, data, etc. provided under control of the processor 3011 may be transmitted to the user terminal 2010 and/or an external device through the communication module 3013 and a network.

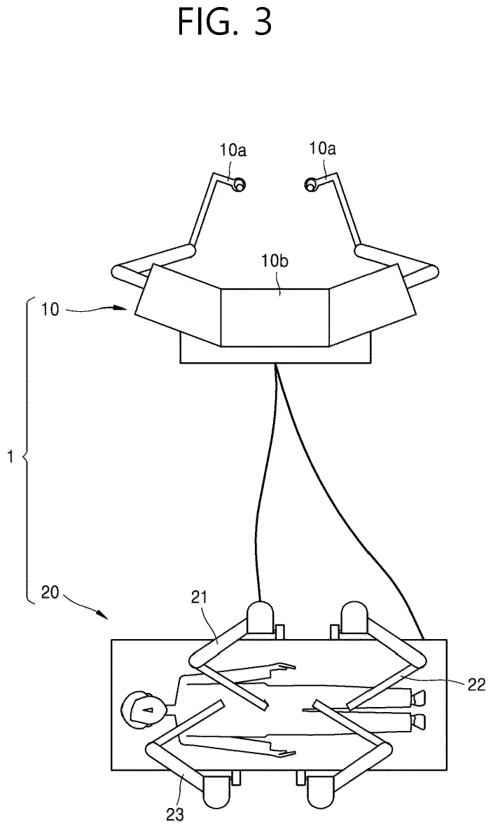

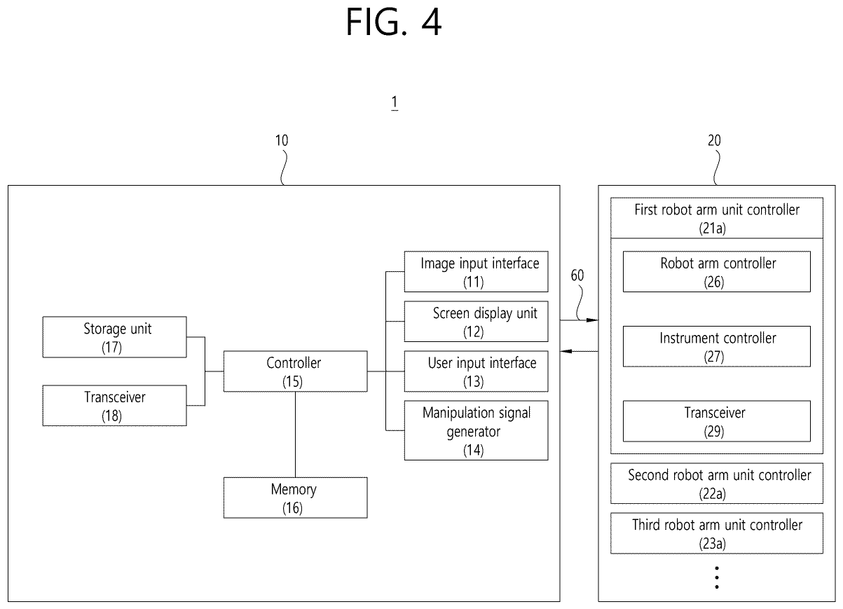

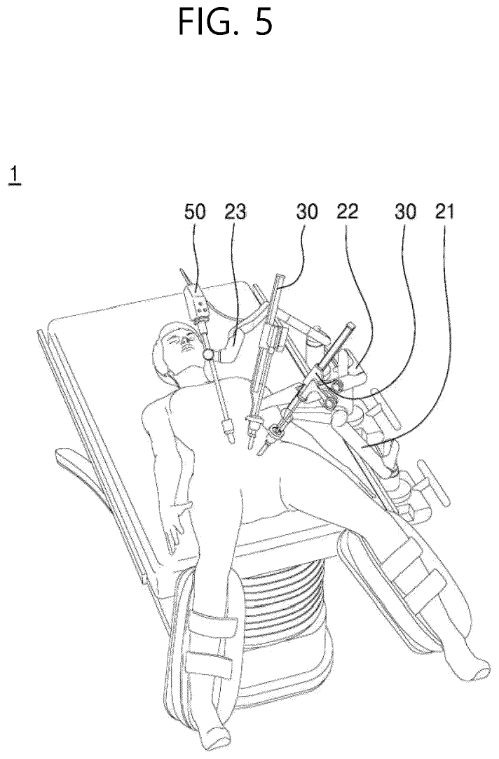

FIG. 3 is a diagram for explaining another example of a surgical robot system that drives a surgical instrument according to an embodiment. FIG. 4 is a block diagram illustrating the internal configuration of the surgical robot system of FIG. 3. FIG. 5 is a perspective view of a slave robot of the surgical robot system of FIG. 3 and a multi-joint surgical instrument mounted thereon.

Referring to FIGS. 3 to 5, a surgical robot system 1 includes a master robot 10, a slave robot 20, and a multi-joint surgical instrument 30.

The master robot 10 includes manipulation members 10a and a display member 10b, and the slave robot 20 includes one or more robot arm units 21, 22, and 23.

The master robot 10 includes the manipulation members 10a so that a surgical operator may grip and manipulate the same respectively with both hands. The manipulation members 10a may be implemented as two or more handles as illustrated in FIG. 3, and manipulation signals according to the handle manipulation of the surgical operator are transmitted to the slave robot 20 through a wired or wireless communication network so that the robot arm units 21, 22, and 23 are controlled. In other words, surgical operations such as positioning, rotation, and cutting work of the robot arm units 21, 22, and 23 may be performed by the handle manipulation of the surgical operator. Herein, the manipulation signal may be, for example, manipulation information generated by a processor, but is not limited thereto.

For example, the surgical operator may manipulate the robot arm units 21, 22, and 23 using manipulation levers in the form of a handle. The manipulation lever as described above may have various mechanical configurations according to the manipulation method thereof, and may be provided in various configurations for operating the robot arm units 21, 22, and 23 of the slave robot 20 and/or other surgical equipment, such as a master handle manipulating the operation of each of the robot arm units 21, 22, and 23 and various input tools added to the master robot 10 for manipulating the functions of the entire system such as joystick, keypad, trackball, foot pedal, and touch screen. Herein, the manipulation member 10a is not limited to the shape of a handle and may be applied without any limitation as long as the manipulation member 10a may control operations of the robot arm units 21, 22, and 23 through a network such as a wired or wireless communication network.

According to an embodiment of the present disclosure, manipulation information may be generated based on the manipulation lever or manipulation member 10a described above. For example, according to an embodiment of the present disclosure, manipulation information may be generated based on the operation of a user manipulating the manipulation lever or manipulation member 10a. However, examples of generating manipulation information are not limited to the above description.

Alternatively, a voice input or a motion input may also be applied as user input. In other words, a user may wear, on the head thereof, glasses or a head mount display (HMD), to which a sensor is attached, and a laparoscope 50 may move according to a direction of the gaze. Alternatively, when the user issues a command with voice, such as “left”, “right”, “first arm”, “second arm”, and the like, the voice command may be recognized and the motion may be performed. For example, an embodiment of the present disclosure may generate manipulation information based on the voice of the user.

An image captured through the laparoscope 50 to be described later is displayed as a screen image on the display member 10b of the master robot 10. In addition, a predetermined fictive manipulation plate may be displayed independently or displayed together with the image captured by the laparoscope 50 on the display member 10b.

The display member 10b may include one or more monitors, each of which may individually display information necessary for surgery. The quantity of monitors may be variously decided depending on the type or kind of information that needs to be displayed.

The slave robot 20 may include one or more robot arm units 21, 22, and 23. Herein, each of the robot arm units 21, 22, and 23 may be provided in the form of a module that may operate independently of each other, and in this connection, an algorithm for preventing a collision between the robot arm units 21, 22, and 23 may be applied to the surgical robot system 1.

In general, a robot arm refers to an apparatus having a function similar to that of the arm and/or the wrist of a human being and having a wrist portion to which a predetermined tool may be attached. In an embodiment of the present disclosure, the robot arm units 21, 22, and 23 may each be defined as a concept encompassing all of the components such as an upper arm, a lower arm, a wrist, and an elbow, a multi-joint surgical instrument coupled to the wrist portion, and the like. Alternatively, the robot arm unit may also be defined as a concept that includes only components for driving the multi-joint surgical instrument, excluding the multi-joint surgical instrument coupled to the wrist portion.

The robot arm units 21, 22, and 23 of the slave robot 20 described above may be implemented to be driven with multiple degrees of freedom. The robot arm units 21, 22, and 23 may include, for example, a surgical instrument inserted into a surgical site of a patient, a yaw driving unit for rotating the surgical instrument in a yaw direction according to a surgical position, a pitch driving unit for rotating the surgical instrument in a pitch direction perpendicular to a rotational driving of the yaw driving unit, a transfer driving unit for moving the surgical instrument in a length direction, a rotation driving unit for rotating the surgical instrument, and a surgical instrument driving unit for incising or cutting the surgical lesion by driving an end effector at an end of the surgical instrument. However, the configuration of the robot arm units 21, 22, and 23 is not limited thereto, and it should be understood that this example does not limit the scope of the present disclosure. Herein, a detailed description of the actual control process, such as rotation and movement of the robot arm units 21, 22, and 23 in a corresponding direction by the surgical operator manipulating the manipulating member 10a, will be omitted.

Herein, two of the robot arm units 21, 22, and 23 may have the multi-joint surgical instrument 30 attached thereto, and one of the robot arm units 21, 22, and 23 may have the laparoscope 50 attached thereto. In addition, a surgical operator may select the robot arm unit 21, 22, or 23 to be controlled via the master robot 10. As described above, by directly controlling a total of three or more surgical instruments through the master robot 10, the surgical operator may accurately and freely control various instruments according to the intention of the surgical operator without a surgical assistant.

One or more slave robots 20 may be provided to operate a patient, and the laparoscope 50 for allowing a surgical site to be displayed as a screen image through the display member 10b may be implemented as an independent slave robot 20. In addition, as described above, the embodiments of the present disclosure may be used universally for surgeries in which various surgical endoscopes other than laparoscopes (for example, thoracoscopic, arthroscopic, rhinoscopic, and the like) are used.

The master robot 10 may perform various activities such as generating manipulation information based on an amount of change in the reference posture of the user input interface for controlling the surgical instrument, deciding the target posture of the surgical instrument corresponding to the manipulation information, deciding the target state information for the driving element based on whether the target posture exceeds the driving limit for the at least one driving element provided in the surgical instrument, or driving the driving element according to the target state information.

For example, the master robot 10 transmits at least one piece of the manipulation information or the target state information of the driving element determined based thereon to the slave robot 20 through a wired or wireless communication network to control the robot arm units 21, 22, and 23. In other words, surgical operations such as positioning, rotation, and cutting work of the robot arm units 21, 22, and 23 may be performed by the handle manipulation of a surgical operator. In other words, when the manipulation information is decided by the master robot 10, the decided manipulation information may be transmitted to the slave robot 20 through a wired or wireless communication network, and the slave robot 20 may decide the target state information based on the manipulation information. According to another aspect, the master robot 10 may decide manipulation information, decide target state information corresponding thereto, and transmit the decided target state information to the slave robot 20.

Referring to FIG. 4, in an embodiment of the present disclosure, the master robot 10 may include an image input interface 11, a screen display unit 12, a user input interface 13, a manipulation signal generator 14, a controller 15, a memory 16, a storage unit 17, and a transceiver 18.

The master robot 10 may be included in the user terminal of FIG. 2A. For example, the manipulation signal generator 14 and the controller 15 may be included in the processor 2011, the memory 16 and the storage unit 17 may be included in the memory 2012, and the transceiver 18 may be included in the communication module 2014, but the example of the master robot 10 is not limited to the above.

The image input interface 11 may receive an image captured by a camera provided in the laparoscope 50 of the slave robot 20 through a wired or wireless communication network. An image captured through a camera may include an image representing the operation of the surgical instrument driven according to target state information.

The screen display unit 12 outputs a screen image corresponding to the image received through the image input interface 11 as visual information. In addition, the screen display unit 12 may further output information corresponding to biometric information of a subject to be treated, when the biometric information is input. In addition, the screen display unit 12 may further output image data (for example, an X-ray image, a CT image, an MRI image, or the like) associated with a patient for a surgical site. Herein, the screen display unit 12 may be implemented in the form of a display member (see 10b of FIG. 3), and an image processing process for allowing the received image to be output as a screen image through the screen display unit 12 may be performed by the controller 15. Herein, the image may include an image representing the operation of the surgical instrument driven according to target state information.

In the embodiment illustrated in FIG. 4, the image input interface and the screen display unit are illustrated as being included in the master robot 10, but an embodiment of the present disclosure is not limited thereto. The display member may be provided as a separate member spaced apart from the master robot 10. Alternatively, the display member may be provided as one component of the master robot 10. In addition, in another embodiment, a plurality of display members may be provided, one of which may be disposed adjacent to the master robot 10, and others thereof may be disposed at some distance from the master robot 10.

Herein, the screen display unit 12 (in other words, the display member 10b of FIG. 3) may be provided as a three-dimensional display apparatus. In detail, the three-dimensional display apparatus refers to an image display apparatus in which depth information is added to a two-dimensional image by applying a stereoscopic technique, and this depth information is used to enable an observer to feel a three-dimensional living feeling and a sense of reality. The surgical robot system 1 according to an embodiment of the present disclosure may provide a more realistic fictive environment to a user by including a three-dimensional display apparatus as the screen display unit 12.

The user input interface 13 is a member for allowing a surgical operator to manipulate the positions and functions of the robot arm units 21, 22, and 23 of the slave robot 20. The user input interface 13 may be formed in the form of a handle-shaped manipulation member (see 10a of FIG. 3) as illustrated in FIG. 3, but the shape thereof is not limited thereto and may be implemented by being modified in various shapes to achieve the same purpose. In addition, for example, some of the user input interface 13 may be formed in the shape of a handle, and the others thereof may be formed in a different shape, such as a clutch button. In addition, a finger insertion tube or insertion ring may be further formed so as to allow the fingers of a surgical operator to be inserted therethrough and fixed to facilitate manipulation of a surgical tool.

According to an embodiment of the present disclosure, manipulation information may be generated based on the operation of a surgical operator on the user input interface 13. For example, according to an embodiment of the present disclosure, manipulation information can be generated based on the operation of the surgical operator manipulating the user input interface 13. However, examples of generating manipulation information are not limited to the above.

The manipulation signal generator 14 generates a corresponding manipulation signal when a surgical operator manipulates the user input interface 13 to move the position of the robot arm units 21, 22, and 23 or manipulate the surgical operation. As an example, the manipulation signal generator 14 may generate corresponding manipulation information when the surgical operator manipulates the user input interface 13 to move the position of the robot arm units 21, 22, and 23 or manipulate the surgical operation.

For example, the manipulation signal generator 14 transmits the generated manipulation signal to the controller 15 or to the slave robot 20 through the transceiver 18. The manipulation signal may be transmitted and received through a wired or wireless communication network. Based on the transmitted manipulation signal, the controller 15 may control the slave robot 20 or the multi-joint surgical instrument 30 to operate. Alternatively, based on the transmitted manipulation signal, a robot arm controller 26 included in the slave robot 20 may control the robot arm units 21, 22, and 23 to operate. Alternatively, based on the transmitted manipulation signal, an instrument controller 27 included in the slave robot 20 may control the multi-joint surgical instrument 30 to operate. However, the method by which the operation of the slave robot 20 or the multi-joint surgical instrument 30 is controlled based on the manipulation signal is not limited to the aforementioned method.

The instrument controller 27 receives the manipulation signal generated by the manipulation signal generator 14 of the master robot 10 and controls the multi-joint surgical instrument 30 to operate according to the manipulation signal.

The controller 15 is a kind of central processing device, and controls the operation of each component so that the aforementioned functions may be performed. In an example, the controller 15 may perform a function of transforming an image input through the image input interface 11 into a screen image to be displayed through the screen display unit 12. As another example, the controller 15 may generate the target posture of the robot arm units 21, 22, and 23 based on manipulation information. In addition, the controller 15 may determine whether the driving limit of the at least one driving element of the robot arm unit 21, 22, or 23 is exceeded based on the target posture. In addition, the controller 15 may decide target state information of the at least one driving element based on the determination result of whether the driving limit is exceeded. In addition, the controller 15 may drive the robot arm units 21, 22, and 23 based on the decided target state information.

According to the above description, it has been described that the controller 15 calculates the target posture based on the manipulation information, determination of whether the driving limit is exceeded, and target state information, which may be performed by other controllers according to an embodiment of the present disclosure (for example, by the robot arm controller 26, or the instrument controller 27), without being limited thereto.

The memory 16 may perform a function of temporarily or permanently storing data processed by the controller 15. Herein, the memory 16 may include a magnetic storage medium or a flash storage medium, but the scope of the present disclosure is not limited thereto.

The storage unit 17 may store data received from the slave robot 20. In addition, the storage unit 17 may store various pieces of input data (for example, patient data, device data, surgery data, and the like).

The transceiver 18 interworks with a communication network 60 to provide a communication interface necessary for transmitting and receiving image data transmitted from the slave robot 20 and control data transmitted from the master robot 10. The image data transmitted from the slave robot 20 may include an image representing the operation of the surgical instrument driven according to target state information. The control data transmitted from the master robot 10 may include at least one piece of manipulation information on an amount of change in the user input interface or target state information on an operation of the slave robot 20.

The slave robot 20 includes a plurality of robot arm unit controllers 21a, 22a, and 23a. In addition, the robot arm unit controller 21a includes a robot arm controller 26, an instrument controller 27, and a transceiver 29.

The robot arm controller 26 may receive a manipulation signal generated by the manipulation signal generator 14 of the master robot 10, and may serve to control the robot arm units 21, 22, and 23 to operate according to the manipulation signal. For example, the robot arm controller 26 may receive manipulation information or target state information calculated from the master robot 10, and may serve to control the robot arm units 21, 22, and 23 to operate accordingly.

The instrument controller 27 may receive a manipulation signal generated by the manipulation signal generator 14 of the master robot 10, and may serve to control the multi-joint surgical instrument 30 to operate according to the manipulation signal. For example, the instrument controller 26 may receive manipulation information or target state information calculated from the master robot 10, and may serve to control the multi-joint surgical instrument 30 to operate accordingly.

The transceiver 29 interworks with the communication network 60 to provide a communication interface necessary for transmitting and receiving image data transmitted from the slave robot 20 and control data transmitted from the master robot 10. The image data transmitted from the slave robot 20 may include an image representing the operation of the surgical instrument driven according to target state information. The control data transmitted from the master robot 10 may include at least one piece of manipulation information on an operation of the slave robot 20 or target state information.

The communication network 60 serves to connect the master robot 10 and the slave robot 20. In other words, the communication network 60 refers to a communication network for providing an access path so that data may be transmitted and received between the master robot 10 and the slave robot 20 after the master robot 10 and the slave robot 20 are connected. The communication network 60 may be, for example, a wired network such as local area networks (LANs), wired area networks (WANs), metropolitan area networks (MANs), and integrated service digital networks (ISDNs), or a wireless network such as wireless LANs, code division multiple access (CDMA), Bluetooth, and satellite communication, but the scope of an embodiment of the present disclosure is not limited thereto.

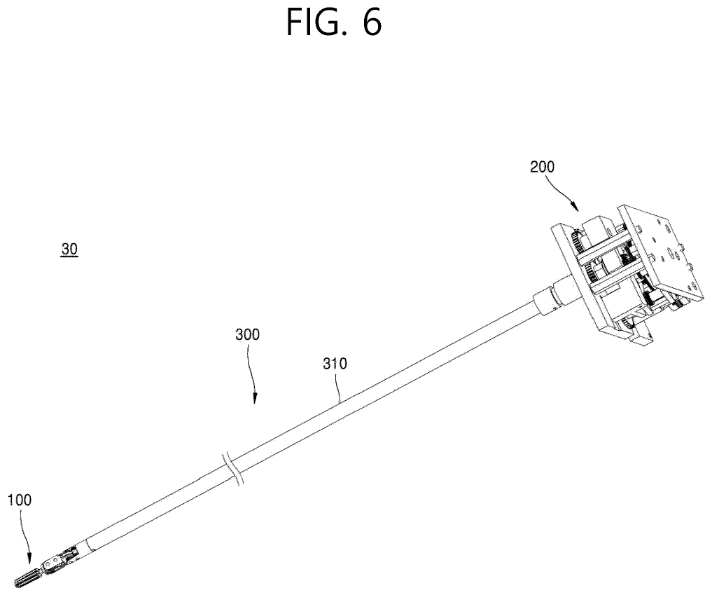

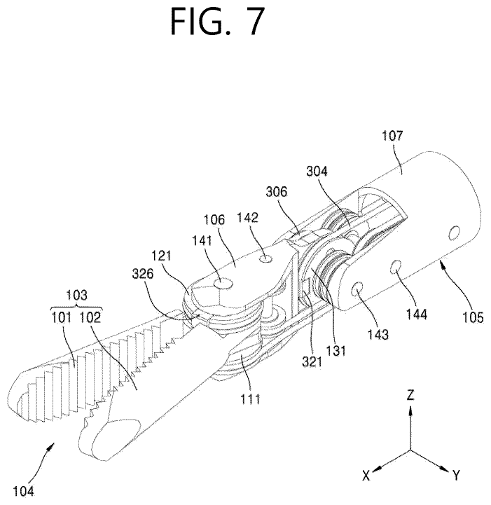

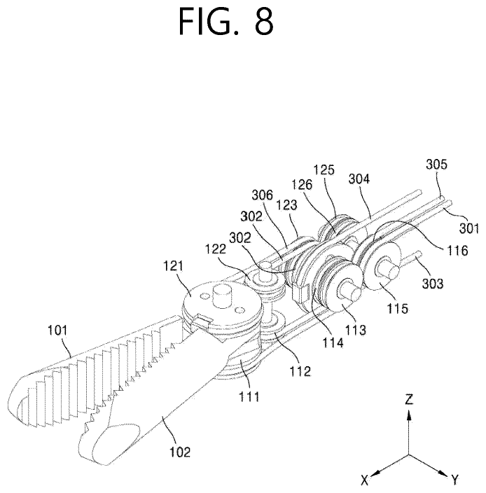

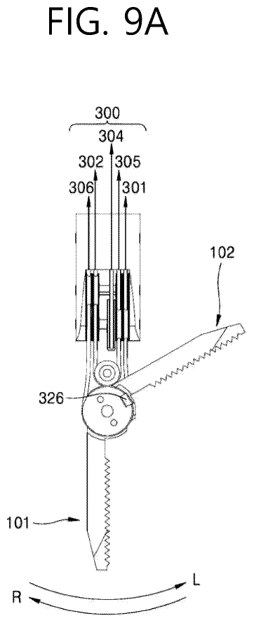

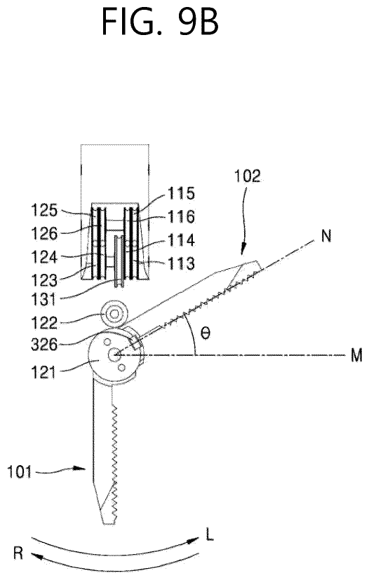

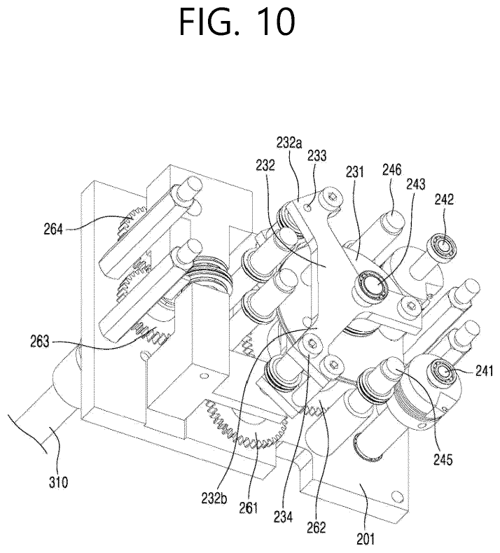

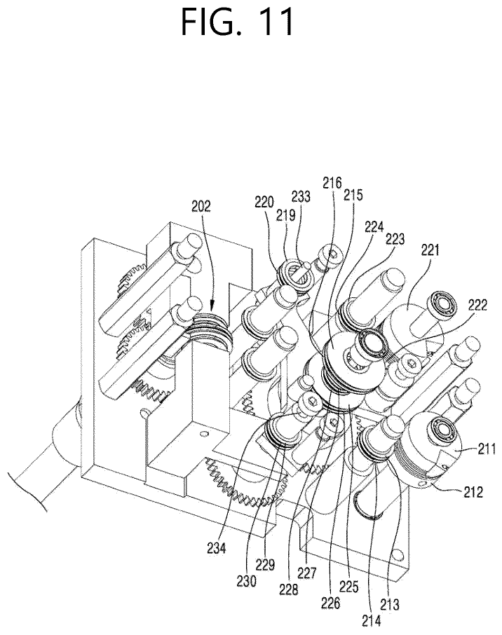

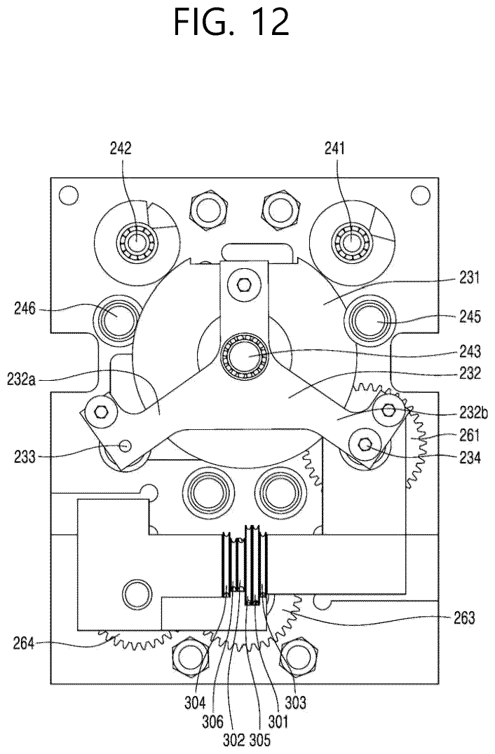

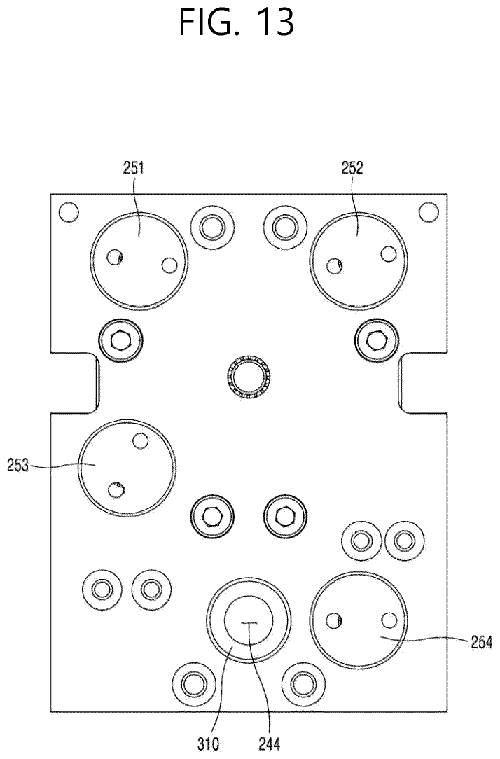

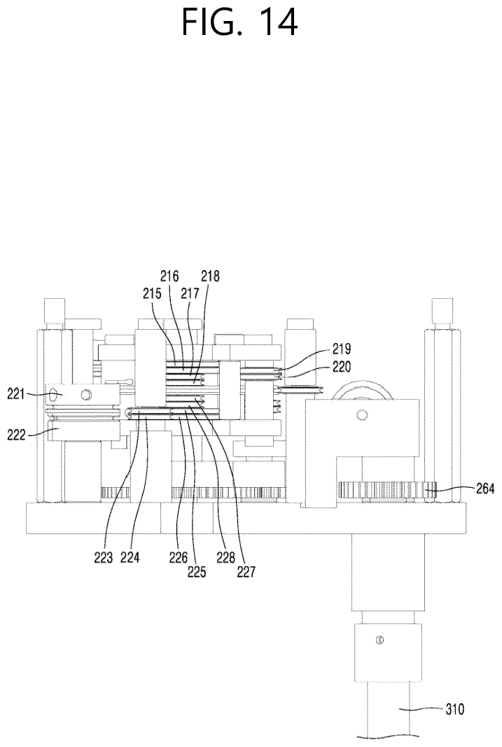

FIG. 6 is a perspective view illustrating a multi-joint type surgical instrument according to an embodiment of the present disclosure, FIGS. 7 and 8 are perspective views of an end tool of the multi-joint type surgical instrument of FIG. 6, and FIGS. 9A to 9B is a plan view of the end tool of the multi-joint type surgical instrument of FIG. 6. FIGS. 10 and 11 are perspective views of a driving part of the multi-joint type surgical instrument of FIG. 6, FIG. 12 is a plan view of the driving part of the multi-joint type surgical instrument of FIG. 6, FIG. 13 is a rear view of the driving part of the multi-joint type surgical instrument of FIG. 6, and FIG. 14 is a side view of the driving part of the multi-joint type surgical instrument of FIG. 6.

Referring first to FIG. 6, the multi-joint type surgical instrument 30 according to an embodiment of the present disclosure may include an end tool 100, a driving part 200, and a power transmission part 300, and the power transmission part 300 may include a connection part 310.

The connection part 310 is formed in the shape of a hollow shaft, in which one or more wires (to be described later) may be accommodated, and may have one end portion to which the driving part 200 is coupled and the other end portion to which the end tool 100 is coupled, and serve to connect the driving part 200 and the end tool 100.

The driving part 200 is formed at one end portion of the connection part 310 and provides an interface capable of being coupled to the robot arm unit (see 21 or the like in FIG. 3). Accordingly, when a user operates the master robot (see 10 in FIG. 3), a motor (not shown) of the robot arm unit (see 21 or the like in FIG. 3) is operated so that the end tool 100 of the multi-joint type surgical instrument 30 can perform a motion corresponding thereto, and a driving force of the motor (not shown) is transmitted to the end tool 100 through the driving part 200. In other words, it may be described that the driving part 200 itself becomes an interface that connects between the multi-joint type surgical instrument 30 and the slave robot 20.

For example, when the user input part 13 (see FIG. 3) is operated by a user, a motor (not shown) of the robot arm unit 21 or the like (see FIG. 3) operates so that the end tool 100 of the multi-joint type surgical instrument 30 can perform a motion corresponding thereto, and a driving force of the motor (not shown) may be transmitted to the end tool 100 through the driving part 200.

The end tool 100 is formed on the other end portion of the connection part 310, and performs necessary motions for surgery by being inserted into a surgical site. In an example of the above-described end tool 100, as shown in FIG. 7, a pair of jaws 101 and 102 for performing a grip motion may be used. However, the embodiment of the present disclosure is not limited thereto, and various devices for performing surgery may be used as the end tool 100. For example, a configuration such as a cantilever cautery may also be used as the end tool. The above-described end tool 100 is connected to the driving part 200 by the power transmission part 300 and receives a driving force through the power transmission part 300 to perform a motion necessary for surgery, such as a gripping motion, a cutting motion, a suturing motion, or the like.

Here, the end tool 100 of the multi-joint type surgical instrument 30 according to an embodiment of the present disclosure is formed to be rotatable in at least two or more directions, for example, the end tool 100 may be formed to perform a pitch motion around a rotation shaft 143 of FIG. 7 and simultaneously perform a yaw motion and an actuation motion around a rotation shaft 141 of FIG. 7.

Here, each of a pitch motion, a yaw motion, an actuation motion, and a roll motion as used in the present disclosure are defined as follows.

First, the pitch motion means a motion of the end tool 100 rotating in a vertical direction with respect to an extension direction of the connection part 310 (an X-axis direction of FIG. 6), that is, a motion rotating around the Y-axis of FIG. 7. In other words, the pitch motion means a motion of the end tool 100, which is formed to extend from the connection part 310 in the extension direction of the connection part 310 (the X-axis direction of FIG. 7), rotating vertically around the Y-axis with respect to the connection part 310.

Next, the yaw motion means a motion of the end tool 100 rotating in left and right directions, that is, a motion rotating around a Z-axis of FIG. 7, with respect to the extension direction of the connection part 310 (the X-axis direction of FIG. 7). In other words, the yaw motion means a motion of the end tool 100, which is formed to extend from the connection part 310 in the extension direction of the connection part 310 (the X-axis direction of FIG. 7), rotating horizontally around the Z-axis with respect to the connection part 310. That is, the yaw motion relates to a motion of two jaws 101 and 102, which are formed on the end tool 100, rotating around the Z-axis in the same direction.

Meanwhile, the actuation motion means a motion of the end tool 100 rotating around the same shaft of rotation as that of the yaw motion, while the two jaws 101 and 102 rotate in the opposite directions so as to be closed or opened. That is, the actuation motion means rotating motions of the two jaws 101 and 102, which are formed on the end tool 100, in the opposite directions around the Z-axis.

Defining this from another perspective, the yaw rotation may be defined as a motion in which an end tool jaw pulley (to be described later) rotates around the rotation shaft 141, which is an end tool jaw pulley rotation shaft, and the pitch rotation may be defined as a motion in which the end tool jaw pulley revolves around the rotation shaft 143, which is an end tool pitch rotation shaft.

The roll motion refers to a motion in which the multi-joint type surgical instrument rotates with the connection part 310 as a shaft. For example, the roll motion may be a motion in which the multi-joint type surgical instrument rotates in the clockwise or counterclockwise direction around the extension direction of the connection part 310 (the X-axis direction of FIG. 7).

Meanwhile, the roll motion may mean a motion in which the end tool 100 rotates around the X-axis with respect to the connection part 310. For example, the roll motion may be a motion in which the end tool rotates in the clockwise or counterclockwise direction around the extension direction of the connection part 310 (the X-axis direction of FIG. 7).

The power transmission part 300 may connect the driving part 200 and the end tool 100, transmit the driving force from the driving part 200 to the end tool 100, and include a plurality of wires, pulleys, links, sections, gears, or the like.

Hereinafter, the end tool 100, the driving part 200, the power transmission part 300, and the like of the multi-joint type surgical instrument 30 of FIG. 6 will be described in more detail.

Hereinafter, the power transmission part 300 of the multi-joint type surgical instrument 30 of FIG. 6 will be described in more detail.

Referring to FIGS. 6 to 14, the power transmission part 300 of the multi-joint type surgical instrument 30 according to an embodiment of the present disclosure may include a plurality of wires 301, 302,303, 304, 305, and 306.

Here, the wires 301 and 305 may be paired to serve as first jaw wires. The wires 302 and 306 may be paired to serve as second jaw wires. Here, the components encompassing the wires 301 and 305, which are first jaw wires, and the wires 302 and 306, which are second jaw wires, may be referred to as jaw wires. In addition, the wires 303 and 304 may be paired to serve as pitch wires.

Here, in the drawings, a pair of wires are illustrated as being associated with a rotational motion of a first jaw 101, and a pair of wires are illustrated as being associated with a rotational motion of a second jaw 102, but an embodiment of the present disclosure is not limited thereto. For example, a pair of wires may be associated with a yaw motion, and a pair of wires may be associated with an actuation motion.

In addition, the power transmission part 300 of the multi-joint type surgical instrument 30 according to an embodiment of the present disclosure may include a coupling member 321, a coupling member 326, and the like, which are coupled to respective end portions of the wires in order to couple the wires and the pulleys. Here, each of the coupling members may have various shapes as necessary, such as a ball shape, a tube shape, and the like.

Here, the coupling member 321, which is a pitch wire coupling member, is coupled to the end portions of the wires 303 and 304, which are pitch wires, at the end tool 100 side to serve as a pitch wire-end tool coupling member. Meanwhile, although not illustrated in the drawings, a pitch wire-driving part coupling member (not shown) may be coupled to the end portions of the wires 303 and 304, which are pitch wires, at the driving part 200 side.

Meanwhile, the coupling member 326, which is a second jaw wire coupling member, is coupled to the end portions of the wires 302 and 306, which are second jaw wires, at the end tool 100 side to serve as a second jaw wire-end tool coupling member. Meanwhile, although not illustrated in the drawings, a second jaw wire-driving part coupling member (not shown) may be coupled to the end portions of the wires 302 and 306, which are second jaw wires, at the driving part 200 side.

Meanwhile, although not illustrated in the drawings, a coupling member (not shown) having the same shape as the second jaw wire coupling member 326 may be coupled to the end portions of the wires 301 and 305, which are first jaw wires, at the end tool 100 side to serve as a first jaw wire-end tool coupling member. Meanwhile, although not illustrated in the drawings, a first jaw wire-driving part coupling member (not shown) may be coupled to the end portions of the wires 301 and 305, which are first jaw wires, at the driving part 200 side.

Here, each of the coupling members is classified as being included in the power transmission part 300, but the coupling members may be classified such that the coupling member at the end tool 100 side may be included in the end tool 100, and the coupling member at the driving part 200 side may be included in the driving part 200.

The coupling relationship between the wires, the fastening members, and the respective pulley will be described in detail as follows.

First, the wires 302 and 306, which are second jaw wires, may be a single wire. The second jaw wire coupling member 326, which is a second jaw wire-end tool coupling member, is inserted at an intermediate point of the second jaw wire, which is a single wire, and the second jaw wire coupling member 326 is crimped and fixed, and then, both strands of the second jaw wire centered on the second jaw wire coupling member 326 may be referred to as the wire 302 and the wire 306, respectively.

Alternatively, the wires 302 and 306, which are second jaw wires, may also be formed as separate wires, and connected to each other by the second jaw wire coupling member 326.

In addition, by coupling the second jaw wire coupling member 326 to a pulley 121, the wires 302 and 306 may be fixedly coupled to the pulley 121. This allows the pulley 121 to rotate as the wires 302 and 306 are pulled and released.

Meanwhile, the second jaw wire-driving part coupling member (not shown) may be coupled to the end portions of the wires 302 and 306, which are opposite to the end portions to which the second jaw wire coupling member 326 is coupled. That is, the second jaw wire-driving part coupling member (not shown) may be fixed to each of the wires 302 and 306 by inserting the opposite end portions of the wires 302 and 306 into the second jaw wire-driving part coupling member (not shown) and crimping the coupling member (not shown).

In addition, by coupling the second jaw wire-driving part coupling member (not shown) coupled to the wires 302 and 306 to each of the pulley 221 and the pulley 222, the wire 302 and the wire 306 may be fixedly coupled to the pulley 221 and the pulley 222, respectively. As a result, when the pulley 221 and the pulley 222 are rotated by a motor or a human force, the pulley 121 of the end tool 100 may be rotated as the wire 302 and the wire 306 are pulled and released.

Here, a driving part second jaw pulley may include two pulleys of the pulley 221 and the pulley 222, and thus the second jaw wire-driving part coupling member may also include two coupling members. Alternatively, the driving part second jaw pulley includes one pulley, the second jaw wire-driving part coupling member also includes one coupling member, and the wires 302 and 306 may be coupled to one coupling member to be coupled to one driving part second jaw pulley.

In the same manner, the wire 301 and the wire 305, which are first jaw wires, are coupled to the first jaw wire-end tool coupling member (not shown) and the first jaw wire-driving part coupling member (not shown), respectively. In addition, the first jaw wire-end tool coupling member (not shown) is coupled to a pulley 111, and the first jaw wire-driving part coupling member (not shown) is coupled to a pulley 211 and a pulley 212. As a result, when the pulleys 211 and 212 are rotated by a motor or a human force, the pulley 111 of the end tool 100 may be rotated as the wire 301 and the wire 305 are pulled and released.

In the same manner, each of one end portions of the wires 303 and 304, which are pitch wires, is coupled to the pitch wire coupling member 321, which is a pitch wire-end tool coupling member, and each of the other end portions of the wires 303 and 304 are coupled to the pitch wire-driving part coupling member (not shown). In addition, the pitch wire coupling member 321 is coupled to a pulley 131, and the pitch wire-driving part coupling member (not shown) is coupled to a pulley 231. As a result, when the pulley 231 is rotated by a motor or a human force, the pulley 131 of the end tool 100 may be rotated as the wire 303 and the wire 304 are pulled and released.

As a result, the wire 301 and the wire 305, which are both strands of the first jaw wire, are coupled to a coupling member 323, which is a first jaw wire-end tool coupling member, and the first jaw wire-driving part coupling member (not shown) so as to form as a whole a closed loop. Similarly, the second jaw wire and the pitch wire may each be formed to form a closed loop.

Hereinafter, the end tool 100 of the multi-joint type surgical instrument 30 of FIG. 6 will be described in more detail.

FIGS. 7 and 8 are perspective views illustrating the end tool of the multi-joint type surgical instrument of FIG. 6, and FIG. 7 is a plan view illustrating the end tool of the multi-joint type surgical instrument of FIG. 6. Here, FIG. 7 illustrates a state in which an end tool hub 106 and a pitch hub 107 are coupled, and FIG. 8 illustrates a state in which the end tool hub 106 and the pitch hub 107 are removed.

Referring to FIGS. 7 to 9, the end tool 100 according to an embodiment of the present disclosure includes a pair of jaws for performing a grip motion, that is, the first jaw 101 and the second jaw 102. Here, each of the first jaw 101 and the second jaw 102, or a component encompassing the first jaw 101 and the second jaw 102 may be referred to as a jaw 103.

Further, the end tool 100 may include the pulley 111, a pulley 112, a pulley 113, a pulley 114, a pulley 115, and a pulley 116 that are related to a rotational motion of the first jaw 101. In addition, the end tool 100 may include the pulley 121, a pulley 122, a pulley 123, a pulley 124, a pulley 125, and a pulley 126 that are related to a rotational motion of the second jaw 102.

Here, in the drawings, one group of pulleys are illustrated as being associated with a rotational motion of the first jaw 101, and one group of pulleys are illustrated as being associated with a rotational motion of the second jaw 102, but an embodiment of the present disclosure is not limited thereto. For example, one group of pulleys in the end tool may be associated with a yaw motion, and one group of pulleys in the end tool may be associated with an actuation motion. Here, the pulleys included in the end tool 100, including the pulleys described above, may be collectively referred to as end tool pulleys.

Meanwhile, the pulleys facing each other are illustrated in the drawings as being formed parallel to each other, but an embodiment of the present disclosure is not limited thereto, and each of the pulleys may be variously formed with a position and a size suitable for the configuration of the end tool.

Further, the end tool 100 according to an embodiment of the present disclosure may include the end tool hub 106 and the pitch hub 107.

The rotation shaft 141 and a rotation shaft 142, which will be described later, may be inserted through the end tool hub 106, and the end tool hub 106 may internally accommodate at least some of the first jaw 101 and the second jaw 102, which are axially coupled to the rotation shaft 141. In addition, the end tool hub 106 may internally accommodate at least some of the pulley 112 and the pulley 122 that are axially coupled to the rotation shaft 142.

In addition, the pulley 131 serving as an end tool pitch pulley may be formed at one end portion of the end tool hub 106. As shown in FIG. 7, the pulley 131 may be formed as a separate member from the end tool hub 106 and coupled to the end tool hub 106. Alternatively, although not illustrated in the drawings, the pulley 131 may be integrally formed with the end tool hub 106 as one body. That is, one end portion of the end tool hub 106 is formed in a disk shape or a semi-circular shape such as a pulley, and a groove around which a wire can be wound may be formed on an outer circumferential surface thereof. The wires 303 and 304 described above are coupled to the pulley 131 serving as an end tool pitch pulley, and a pitch motion may be performed as the pulley 131 is rotated around the rotation shaft 143.

The rotation shaft 143 and a rotation shaft 144, which will be described later, may be inserted through the pitch hub 107, and the pitch hub 107 may be axially coupled to the end tool hub 106 and the pulley 131 by the rotation shaft 143. Thus, the end tool hub 106 and the pulley 131 (coupled thereto) may be formed to be rotatable around the rotation shaft 143 with respect to the pitch hub 107.

Further, the pitch hub 107 may internally accommodate at least some of the pulley 113, the pulley 114, the pulley 123, and the pulley 124 that are axially coupled to the rotation shaft 143. In addition, the pitch hub 107 may internally accommodate at least some of the pulley 115, the pulley 116, the pulley 125, and the pulley 126 that are axially coupled to the rotation shaft 144.

Further, the end tool 100 according to an embodiment of the present disclosure may include the rotation shaft 141, the rotation shaft 142, the rotation shaft 143, and the rotation shaft 144. As described above, the rotation shaft 141 and the rotation shaft 142 may be inserted through the end tool hub 106, and the rotation shaft 143 and the rotation shaft 144 may be inserted through the pitch hub 107.

The rotation shaft 141, the rotation shaft 142, the rotation shaft 143, and the rotation shaft 144 may be arranged sequentially from a distal end 104 of the end tool 100 toward a proximal end 105 thereof. Accordingly, starting from the distal end 104, the rotation shaft 141 may be referred to as a first pin, the rotation shaft 142 may be referred to as a second pin, the rotation shaft 143 may be referred to as a third pin, and the rotation shaft 144 may be referred to as a fourth pin.

Here, the rotation shaft 141 may function as an end tool jaw pulley rotation shaft, the rotation shaft 142 may function as an end tool jaw auxiliary pulley rotation shaft, the rotation shaft 143 may function as an end tool pitch rotation shaft, and the rotation shaft 144 may function as an end tool pitch auxiliary rotation shaft of the end tool 100.

Each of the rotation shafts 141, 142, 143, and 144 may be fitted into one or more pulleys, which will be described in detail below.

The pulley 111 functions as an end tool first jaw pulley, and the pulley 121 functions as an end tool second jaw pulley, and these two components may be collectively referred to as end tool jaw pulleys.

The pulley 111 and the pulley 121, which are end tool jaw pulleys, are formed to face each other, and are formed to be rotatable independently of each other around the rotation shaft 141, which is an end tool jaw pulley rotation shaft. Here, in the drawings, it is illustrated that the pulley 111 and the pulley 121 are formed to rotate around one rotation shaft 141, but it is of course possible that each end tool jaw pulley may be formed to be rotatable around a separate shaft. Here, the first jaw 101 may be fixedly coupled to the pulley 111 and rotated together with the pulley 111, and the second jaw 102 may be fixedly coupled to the pulley 121 and rotated together with the pulley 121. Yaw and actuation motions of the end tool 100 are performed according to the rotation of the pulley 111 and the pulley 121. That is, when the pulley 111 and the pulley 121 are rotated in the same direction around the rotation shaft 141, the yaw motion is performed, and when the pulley 111 and the pulley 121 are rotated in opposite directions around the rotation shaft 141, the actuation motion is performed.

Here, the first jaw 101 and the pulley 111 may be formed as separate members and coupled to each other, or the first jaw 101 and the pulley 111 may be integrally formed as one body. Similarly, the second jaw 102 and the pulley 121 may be formed as separate members and coupled to each other, or the second jaw 102 and the pulley 121 may be integrally formed as one body.

The pulley 112 functions as an end tool first jaw auxiliary pulley, and the pulley 122 functions as an end tool second jaw auxiliary pulley, and these two components may be collectively referred to as end tool jaw auxiliary pulleys.

Specifically, the pulley 112 and the pulley 122, which are end tool jaw auxiliary pulleys, may be additionally provided on one side of the pulley 111 and one side of the pulley 121, respectively. In other words, the pulley 112, which is an auxiliary pulley, may be disposed between the pulley 111 and the pulley 113/pulley 114. In addition, the pulley 122, which is an auxiliary pulley, may be disposed between the pulley 121 and the pulley 123/pulley 124. The pulley 112 and the pulley 122 may be formed to be rotatable independently of each other around the rotation shaft 142. Here, in the drawings, it is illustrated that the pulley 112 and the pulley 122 are formed to rotate around one rotation shaft 142, but it is of course possible that each of the pulley 112 and the pulley 122 may be formed to be rotatable around a separate shaft. Such auxiliary pulleys will be described in more detail later.

The pulley 113 and the pulley 114 function as end tool first jaw pitch main pulleys, and the pulley 123 and the pulley 124 function as end tool second jaw pitch main pulleys, and these two components may be collectively referred to as end tool jaw pitch main pulleys.

The pulley 115 and the pulley 116 function as end tool first jaw pitch sub-pulleys, and the pulley 125 and the pulley 126 function as end tool second jaw pitch sub-pulleys, and these two components may be collectively referred to as end tool jaw pitch sub-pulleys.

Hereinafter, components related to the rotation of the pulley 111 will be described.

The pulley 113 and the pulley 114 function as end tool first jaw pitch main pulleys. That is, the pulley 113 and the pulley 114 function as main rotation pulleys for a pitch motion of the first jaw 101. Here, the wire 301, which is a first jaw wire, is wound around the pulley 113, and the wire 305, which is a first jaw wire, is wound around the pulley 114.

The pulley 115 and the pulley 116 function as end tool first jaw sub-pulleys. That is, the pulley 115 and the pulley 116 function as sub rotation pulleys for a pitch motion of the first jaw 101. Here, the wire 301, which is a first jaw wire, is wound around the pulley 115, and the wire 305, which is a first jaw wire, is wound around the pulley 116.

Here, the pulley 113 and the pulley 114 are disposed on one side of the pulley 111 and the pulley 112 to face each other. Here, the pulley 113 and the pulley 114 are formed to be rotatable independently of each other around the rotation shaft 143 that is an end tool pitch rotation shaft. In addition, the pulley 115 and the pulley 116 are disposed on one side of the pulley 113 and on one side of the pulley 114, respectively, to face each other. Here, the pulley 115 and the pulley 116 are formed to be rotatable independently of each other around the rotation shaft 144 that is an end tool pitch auxiliary rotation shaft. Here, in the drawings, it is illustrated that the pulley 113, the pulley 115, the pulley 114, and the pulley 116 are all formed to be rotatable around a Y-axis direction, but an embodiment of the present disclosure is not limited thereto, and the rotation axes of the respective pulleys may be formed in various directions according to configurations thereof.

The wire 301, which is a first jaw wire, is sequentially wound to make contact with at least portions of the pulley 115, the pulley 113, and the pulley 111. In addition, the wire 305 connected to the wire 301 by the first jaw wire-end tool coupling member 323 is sequentially wound to make contact with at least portions of the pulley 111, the pulley 112, the pulley 114, and the pulley 116 in turn.

Viewed from another perspective, the wires 301 and 305, which are first jaw wires, are sequentially wound to make contact with at least portions of the pulley 115, the pulley 113, the pulley 111, the pulley 112, the pulley 114, and the pulley 116 and are formed to move along the above pulleys while rotating the above pulleys.

Accordingly, when the wire 301 is pulled in the direction of an arrow 301 of FIGS. 9A to 9B, a coupling member (not shown) to which the wire 301 is coupled and the pulley 111 coupled to the coupling member (not shown) are rotated in an arrow L direction of FIGS. 9A to 9B. In contrast, when the wire 305 is pulled in the direction of an arrow 305 of FIGS. 9A to 9B, a coupling member (not shown) to which the wire 305 is coupled and the pulley 111 coupled to the coupling member (not shown) are rotated in an arrow R direction of FIGS. 9A to 9B.

Hereinafter, the pulley 112 and the pulley 122 serving as auxiliary pulleys will be described in more detail.

The pulley 112 and the pulley 122 may serve to increase rotation angles of the first jaw 101 and the second jaw 102, respectively, by coming into contact with the wire 305, which is a first jaw wire, and the wire 302, which is a second jaw wire, and changing the arrangement paths of the wires 305 and 302 to a certain extent.

That is, when the auxiliary pulleys are not disposed, each of the first jaw and the second jaw may be rotated up to a right angle, but in an embodiment of the present disclosure, the pulley 112 and the pulley 122, which are auxiliary pulleys, are additionally provided, so that the maximum rotation angle may be increased by 0 as shown in FIGS. 9A to 9B. This enables a motion of the two jaws of the end tool 100 being opened for an actuation motion while the two jaws are yaw-rotated by 900 in the L direction. This is because the second jaw 102 is rotated by the additional angle θ as shown in FIGS. 9A to 9B. Similarly, an actuation motion is possible even when the two jaws are yaw-rotated in the R direction. In other words, a feature of increasing the range of yaw rotation in which an actuation motion is possible may be obtained through the pulley 112 and the pulley 122.

This will be described in more detail as follows.

When the auxiliary pulleys are not disposed, since the first jaw wire is fixedly coupled to the end tool first jaw pulley, and the second jaw wire is fixedly coupled to the end tool second jaw pulley, each of the end tool first jaw pulley and the end tool second jaw pulley may be rotated up to 90°. In this case, when the actuation motion is performed while the first jaw and the second jaw are located at a 90° line, the first jaw may be opened, but the second jaw may not be rotated beyond 90°. Accordingly, when the first jaw and the second jaw perform a yaw motion over a certain angle, there was a problem that the actuation motion is not smoothly performed.

In order to address such a problem, in the multi-joint type surgical instrument 30 according to an embodiment of the present disclosure, the pulley 112 and the pulley 122, which are auxiliary pulleys, are additionally disposed at one side of the pulley 111 and one side of the pulley 121, respectively. As described above, as the arrangement paths of the wire 305, which is a first jaw wire, and the wire 302, which is a second jaw wire, are changed to a certain extent by disposing the pulley 112 and the pulley 122, a tangential direction of the wires 305 and 302 is changed, and accordingly, the second jaw wire coupling member 326 for coupling the wire 302 and the pulley 121 may be rotated up to a line N of FIGS. 9A to 9B. That is, the second jaw wire coupling member 326, which is a coupling part of the wire 302 and the pulley 121, is rotatable until the second jaw wire coupling member 326 is located on a common internal tangent of the pulley 121 and the pulley 122. Similarly, the first jaw wire-end tool coupling member 323, which is a coupling part of the wire 305 and the pulley 111, is rotatable until the first jaw wire-end tool coupling member 323 is located on a common internal tangent of the pulley 111 and the pulley 112, so that the range of rotation in the L direction may be increased.

In other words, by the pulley 112, the wires 301 and 305, which are two strands of the first jaw wire wound around the pulley 111, are disposed at one side with respect to a plane perpendicular to the Y-axis and passing through the X-axis. Simultaneously, by the pulley 122, the wires 302 and 306, which are two strands of the second jaw wire wound around the pulley 121, are disposed at the other side with respect to the plane perpendicular to the Y-axis and passing through the X-axis.

In other words, the pulley 113 and the pulley 114 are disposed at one side with respect to the plane perpendicular to the Y-axis and passing through the X-axis, and the pulley 123 and the pulley 124 are disposed at the other side with respect to the plane perpendicular to the Y-axis and passing through the X-axis.

In other words, the wire 305 is located on the internal tangent of the pulley 111 and the pulley 112, and the rotation angle of the pulley 111 is increased by the pulley 112. In addition, the wire 302 is located on the internal tangent of the pulley 121 and the pulley 122, and the rotation angle of the pulley 121 is increased by the pulley 122.

According the above-described embodiment of the present disclosure, as the rotation radii of the jaw 101 and the jaw 102 increase, an effect of increasing a yaw motion range in which a normal opening/closing actuation motion is performed may be obtained.

Next, components related to the rotation of the pulley 121 will be described.

The pulley 123 and the pulley 124 function as end tool second jaw pitch main pulleys. That is, the pulley 123 and the pulley 124 function as main rotation pulleys for a pitch motion of the second jaw 102. Here, the wire 306, which is a second jaw wire, is wound around the pulley 123, and the wire 302, which is a second jaw wire, is wound around the pulley 124.

The pulley 125 and the pulley 126 function as end tool second jaw sub-pulleys. That is, the pulley 125 and the pulley 126 function as sub rotation pulleys for a pitch motion of the second jaw 102. Here, the wire 306, which is a second jaw wire, is wound around the pulley 125, and the wire 302, which is a second jaw wire, is wound around the pulley 126.

On one side of the pulley 121, the pulley 123 and the pulley 124 are disposed to face each other. Here, the pulley 123 and the pulley 124 are formed to be rotatable independently of each other around the rotation shaft 143 that is an end tool pitch rotation shaft. In addition, the pulley 125 and the pulley 126 are disposed on one side of the pulley 123 and one side of the pulley 124, respectively, to face each other. Here, the pulley 125 and the pulley 126 are formed to be rotatable independently of each other around the rotation shaft 144, which is an end tool pitch auxiliary rotation shaft. Here, in the drawings, it is illustrated that all of the pulley 123, the pulley 125, the pulley 124, and the pulley 126 are formed to be rotatable around the Y-axis direction, but an embodiment of the present disclosure is not limited thereto, and the rotation axes of the respective pulleys may be formed in various directions according to configurations thereof.

The wire 306, which is a second jaw wire, is sequentially wound to make contact with at least portions of the pulley 125, the pulley 123, and the pulley 121. In addition, the wire 302 connected to the wire 306 by the second jaw wire coupling member 326 is sequentially wound to make contact with at least portions of the pulley 121, the pulley 122, the pulley 124, and the pulley 126.

Viewed from another perspective, the wires 306 and 302, which are second jaw wires, are sequentially wound to make contact with at least portions of the pulley 125, the pulley 123, the pulley 121, the pulley 122, the pulley 124, and the pulley 126, and are formed to move along the above pulleys while rotating the above pulleys.

Accordingly, when the wire 306 is pulled in the direction of an arrow 306 of FIGS. 9A to 9B, the second jaw wire coupling member 326 to which the wire 306 is coupled and the pulley 121 coupled to the second jaw wire coupling member 326 are rotated in the arrow R direction of FIGS. 9A to 9B. In contrast, when the wire 302 is pulled in the direction of an arrow 302 of FIGS. 9A to 9B, the second jaw wire coupling member 326 to which the wire 302 is coupled and the pulley 121 coupled to the second jaw wire coupling member 326 are rotated in the arrow L direction of FIGS. 9A to 9B.

Hereinafter, a pitch motion of the present disclosure will be described in more detail.

First, for the pitch motion, at the end tool 100 side, the pulley 113, the pulley 114, the pulley 123, and the pulley 124, which are end tool jaw pitch main pulleys, are formed to be rotatable around the rotation shaft 143. Meanwhile, in a direction of the proximal end 105 of the end tool jaw pitch main pulley, the pulley 115, the pulley 116, the pulley 125, and the pulley 126, which are end tool jaw pitch sub-pulleys, are formed to be rotatable around the rotation shaft 144.

In addition, based on a plane perpendicular to the rotation shaft 141 and including the rotation shaft 143 (i.e., an XY plane), the wires 301 and 305, which are two strands of the first jaw wire, are located on the same side with respect to the XY plane. That is, the wire 301 and the wire 305 are formed to pass through lower sides of the pulley 113 and the pulley 114, which are end tool jaw pitch main pulleys, and upper sides of the pulley 115 and the pulley 116, which are end tool jaw pitch sub-pulleys.

Similarly, the wires 302 and 306, which are two strands of the second jaw wire, are located on the same side with respect to the XY plane. That is, the wires 302 and 306 are formed to pass through upper sides of the pulley 123 and the pulley 124, which are end tool jaw pitch main pulleys, and lower sides of the pulley 125 and the pulley 126, which are end tool jaw pitch sub-pulleys.

In addition, in the wires 301 and 305 that are two strands of the first jaw wire, when the wire 301 is pulled toward the arrow 301 of FIGS. 9A to 9B and simultaneously the wire 305 is pulled toward the arrow 305 of FIGS. 9A to 9B (i.e., when both strands of the first jaw wire are pulled in the same direction), as shown in FIG. 7, since the wires 301 and 305 are wound around lower portions of the pulleys 113 and 114, which are rotatable around the rotation shaft 143 that is an end tool pitch rotation shaft, the pulley 111 to which the wire 301 and the wire 305 are fixedly coupled, and the end tool hub 106 to which the pulley 111 is coupled are rotated together as a whole in a counterclockwise direction around the rotation shaft 143, as a result, the end tool 100 performs the pitch motion while rotating downward. At this time, since the second jaw 102 and the wires 302 and 306 fixedly coupled thereto are wound around the upper portions of the pulleys 123 and 124 rotatable around the rotation shaft 143, the wires 302 and 306 are unwound in opposite directions of the arrows 302 and 306, respectively.

In contrast, in the wires 302 and 306 that are two strands of the second jaw wire, when the wire 302 is pulled toward the arrow 302 of FIGS. 9A to 9B and simultaneously the wire 306 is pulled toward the arrow 306 of FIGS. 9A to 9B (i.e., when both strands of the second jaw wire are pulled in the same direction), as shown in FIG. 7, since the wires 302 and 306 are wound around lower portions of the pulleys 123 and 124, which are rotatable around the rotation shaft 143 that is an end tool pitch rotation shaft, the pulley 121 to which the wire 302 and the wire 306 are fixedly coupled, and the end tool hub 106 to which the pulley 121 is coupled are rotated together as a whole in a clockwise direction around the rotation shaft 143. As a result, the end tool 100 performs the pitch motion while rotating upward. At this time, since the first jaw 101 and the wires 301 and 305 fixedly coupled thereto are wound around the lower portions of the pulleys 113 and 114 rotatable around the rotation shaft 143, the wires 302 and 306 are moved in opposite directions of the arrows 301 and 305, respectively.

Viewed from another perspective, it may be also described that both strands of each jaw wire are moved simultaneously in the same direction when the end tool 100 is pitch-rotated.

Meanwhile, the end tool 100 of the multi-joint type surgical instrument 30 of the present disclosure may further include the pulley 131, which is an end tool pitch pulley, the driving part 200 may further include the pulley 231, which is a driving part pitch pulley, and the power transmission part 300 may further include the wire 303 and the wire 304 that are pitch wires. Specifically, the pulley 131 of the end tool 100 is rotatable around the rotation shaft 143, which is an end tool pitch rotation shaft, and may be integrally formed with the end tool hub 106 (or fixedly coupled to the end tool hub 106) as one body. In addition, the wires 303 and 304 may serve to connect the pulley 131 of the end tool 100 to the pulley 231 of the driving part 200.

Thus, when the pulley 231 of the driving part 200 is rotated, the rotation of the pulley 231 is transmitted to the pulley 131 of the end tool 100 via the wires 303 and 304, which causes the pulley 131 to also be rotated, and as a result, the end tool 100 performs a pitch motion while rotating.

That is, in the multi-joint type surgical instrument 30 according to an embodiment of the present disclosure, by providing the pulley 131 of the end tool 100, the pulley 231 of the driving part 200, and the wires 303 and 304 of the power transmission part 300 to transmit power for a pitch motion, the driving force for a pitch motion from the driving part 200 may be more completely transmitted to the end tool 100, thereby improving operation reliability.

Here, a diameter of each of the pulley 113, the pulley 114, the pulley 123, and the pulley 124, which are end tool jaw pitch main pulleys, and a diameter of the pulley 131, which is an end tool pitch pulley, may be the same as each other or different from each other. At this time, a ratio of the diameter of the end tool jaw pitch main pulley to the diameter of the end tool pitch pulley may be the same as a ratio of a diameter of a driving part relay pulley of the driving part 200, which will be described later, to a diameter of a driving part pitch pulley 231. This will be described in detail later.

Hereinafter, the driving part 200 of the multi-joint type surgical instrument 30 of FIG. 6 will be described in more detail.

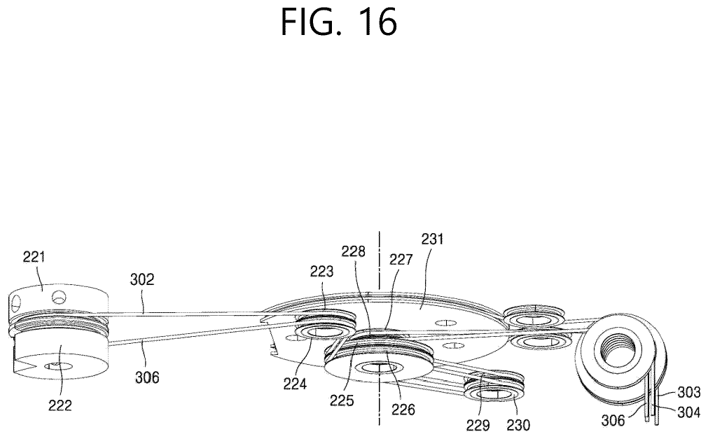

Referring to FIGS. 10 to 16, the driving part 200 of the multi-joint type surgical instrument 30 according to an embodiment of the present disclosure may include the pulley 211, the pulley 212, a pulley 213, a pulley 214, a pulley 215, a pulley 216, a pulley 217, a pulley 218, a pulley 219, and a pulley 220, which are related to a rotational motion of the first jaw 101 In addition, the driving part 200 may include the pulley 221, the pulley 222, a pulley 223, a pulley 224, a pulley 225, a pulley 226, a pulley 227, a pulley 228, a pulley 229, and a pulley 230, which are related to a rotational motion of the second jaw 102.

Here, the pulleys facing each other are illustrated in the drawings as being formed parallel to each other, but an embodiment of the present disclosure is not limited thereto, and each of the pulleys may be variously formed with a position and a size suitable for the configuration of the driving part.

In addition, the driving part 200 of the multi-joint type surgical instrument 30 according to an embodiment of the present disclosure may further include the pulley 231 serving as a driving part pitch pulley, and a pitch-yaw connector 232 configured to connect the pulley 231 to the above-described jaw pulleys of the driving part.

Further, the driving part 200 according to an embodiment of the present disclosure may include a rotation shaft 241, a rotation shaft 242, a rotation shaft 243, a rotation shaft 244, a rotation shaft 245, and a rotation shaft 246. Here, the rotation shaft 241 may function as a first jaw rotation shaft of the driving part, and the rotation shaft 242 may function as a second jaw rotation shaft of the driving part. In addition, the rotation shaft 243 may function as a driving part pitch rotation shaft, and the rotation shaft 244 may function as a driving part roll rotation shaft. In addition, the rotation shaft 245 may function as a driving part first jaw auxiliary rotation shaft of the driving part, and the rotation shaft 246 may function as a driving part second jaw auxiliary rotation shaft. Each of the rotation shafts 241, 242, 243, 244, 245, and 246 may be fitted into one or more pulleys, which will be described in detail later.

In addition, the driving part 200 according to an embodiment of the present disclosure may include a motor coupling part 251, a motor coupling part 252, a motor coupling part 253, and a motor coupling part 254. Here, the motor coupling part 251 may function as a first jaw driving motor coupling part, the motor coupling part 252 may function as a second jaw driving motor coupling part, the motor coupling part 253 may function as a pitch driving motor coupling part, and the motor coupling part 254 may function as a roll driving motor coupling part. Here, each of the motor coupling parts 251, 252, 253, and 254 may be provided in the form of a rotatable flat plate, in which one or more coupling holes, to which a motor (not shown) may be coupled, may be formed.

The motor coupling parts 251, 252, 253, and 254 of the driving part 200 described above are coupled to motors (not shown) formed in the robot arm units 21, 22, and 23, respectively, so that the driving part 200 is operated by driving the motors (not shown).

In addition, the driving part 200 according to an embodiment of the present disclosure may include a gear 261, a gear 262, a gear 263, and a gear 264. Here, the gear 261 and the gear 262 may function as pitch driving gears, and the gear 263 and the gear 264 may function as roll driving gears.

Hereinafter, each component will be described in more detail.

The pulley 211 and the pulley 212 may function as driving part first jaw pulleys, and the pulley 221 and the pulley 222 may function as driving part second jaw pulleys, and these components may be collectively referred to as driving part jaw pulleys.

Here, it is illustrated in the drawings that the pulley 211 is associated with a rotational motion of the first jaw 101 of the end tool 100, and the pulley 221 is associated with a rotational motion of the second jaw 102 of the end tool 100, but an embodiment of the present disclosure is not limited thereto. For example, one group of pulleys in the driving part may be associated with a yaw motion, and one group of pulleys in the driving part may be associated with an actuation motion. Thus, the pulley 211 and the pulley 212 may be collectively referred to as driving part driving pulleys. In addition, in the other pulleys, one group of pulleys may also be associated with a yaw motion, and one group of pulleys may also be associated with an actuation motion.

The pulley 213 and the pulley 214 may function as driving part first jaw auxiliary pulleys, and the pulley 223 and the pulley 224 may function as driving part second jaw auxiliary pulleys, and these components may be collectively referred to as driving part auxiliary pulleys.

The pulley 215 and the pulley 216 may function as driving part first jaw first relay pulleys, and the pulley 217 and the pulley 218 may function as driving part first jaw second relay pulleys, and these components may be collectively referred to as driving part first jaw relay pulleys. Meanwhile, the pulley 225 and the pulley 226 may function as driving part second jaw first relay pulleys, and the pulley 227 and the pulley 228 may function as driving part second jaw second relay pulleys, and these components may be collectively referred to as driving part second jaw relay pulleys. Meanwhile, the pulley 215, the pulley 216, the pulley 225, and the pulley 226 may be collectively referred to as driving part first relay pulleys, and the pulley 217, the pulley 218, the pulley 227, and the pulley 228 may be collectively referred to as driving part second relay pulleys. Furthermore, the pulley 215, the pulley 216, the pulley 217, the pulley 218, the pulley 225, the pulley 226, the pulley 227, and the pulley 228 may be collectively referred to as driving part relay pulleys.

Here, it is illustrated in the drawings that two pulleys are paired to form the driving part relay pulleys for each jaw, but an embodiment of the present disclosure is not limited thereto. For example, it is illustrated that the pulley 215, which is a driving part first jaw first relay pulley, and the pulley 217, which is a driving part first jaw second relay pulley, are formed as a pair, and the wire 301 sequentially passes through the pulley 215 and the pulley 217. However, the driving part first jaw relay pulley may be configured with not just two pulleys but also with three or more pulleys.

Meanwhile, the pulley 219 and the pulley 220 may function as driving part first jaw satellite pulleys, and the pulley 229 and the pulley 230 may function as driving part second jaw satellite pulleys, and these two components may be collectively referred to as driving part satellite pulleys.

A plurality of rotation shafts including the driving part first jaw rotation shaft 241, the driving part second jaw rotation shaft 242, the driving part pitch rotation shaft 243, the driving part roll rotation shaft 244, the driving part first jaw auxiliary rotation shaft 245, and the driving part second jaw auxiliary rotation shaft 246 may be formed on a first surface of a base plate 201. In addition, a plurality of relay pulleys 202 are formed on the first surface of the base plate 201, and may serve to redirect the wires 301, 302, 303, 304, 305, and 306 entering the driving part 200 through the connection part 310 toward the pulley 231.

Further, the connection part 310 in the form of a shaft is coupled to a second surface of the base plate 201 opposite to the first surface, and the first jaw motor coupling part 251, the second jaw driving motor coupling part 252, the pitch driving motor coupling part 253, and the roll driving motor coupling part 254, to which the motors (not shown) for driving the pulleys are coupled, may be formed on the second surface.

Here, each rotation shaft and each motor coupling part may be directly connected or indirectly connected to each other via a gear.

In an example, by directly coupling the first jaw motor coupling part 251 to the driving part first jaw rotation shaft 241, when the first jaw motor coupling part 251 coupled to a first jaw driving motor (not shown) is rotated, the driving part first jaw rotation shaft 241 directly coupled to the first jaw motor coupling part 251 may be rotated together. Similarly, by directly coupling the second jaw driving motor coupling part 252 to the driving part second jaw rotation shaft 242, when the second jaw driving motor coupling part 252 coupled to a second jaw driving motor (not shown) is rotated, the driving part second jaw rotation shaft 242 directly coupled to the second jaw driving motor coupling part 252 may be rotated together.

In another example, when viewed from a plane perpendicular to the driving part pitch rotation shaft 243, the pitch driving motor coupling part 253 and the driving part pitch rotation shaft 243 may be disposed to be spaced apart from each other by a certain extent. In addition, the pitch driving motor coupling part 253 and the driving part pitch rotation shaft 243 may be connected to each other by the gears 261 and 263, which are pitch driving gears.

Similarly, when viewed from a plane perpendicular to the driving part roll rotation shaft 244, the roll driving motor coupling part 254 and the driving part roll rotation shaft 244 may be disposed to be spaced apart from each other by a certain extent. In addition, the roll driving motor coupling part 254 and the driving part roll rotation shaft 244 may be connected to each other by the gears 263 and 264, which are roll driving gears.

As such, some motor coupling parts are configured to be directly connected to the rotation shafts, respectively, and the remaining motor coupling parts are configured to be indirectly connected to the rotation shafts, respectively, because the coupling position and direction between the multi-joint type surgical instrument 30 and the slave robot 20 should be considered. That is, the rotation shaft that is not affected by the coupling position with the slave robot 20 is directly connected to the motor coupling part, whereas the rotation shaft that may cause interference with the coupling position with the slave robot 20 may be indirectly connected to the motor coupling part.

It is illustrated in the drawings that the first jaw motor coupling part 251 and the second jaw driving motor coupling part 252 are directly connected to the rotation shafts, respectively, and the pitch driving motor coupling part 253 and the roll driving motor coupling part 254 are indirectly connected, respectively, through the gears, but an embodiment of the present disclosure is not limited thereto, and various configurations are possible according to the coupling position and direction with the slave robot 20.

The pulleys 211 and 212, which are driving part first jaw pulleys, may be coupled to the driving part first jaw rotation shaft 241. Here, the pulleys 211 and 212 may be formed to rotate together with the driving part first jaw rotation shaft 241.

In addition, the driving part first jaw auxiliary rotation shaft 245 may be disposed in a region adjacent to the driving part first jaw rotation shaft 241. The pulleys 213 and 214, which are driving part first jaw auxiliary pulleys, may be coupled to the driving part first jaw auxiliary rotation shaft 245. Here, the pulleys 213 and 214 may be formed to be rotatable around the driving part first jaw auxiliary rotation shaft 245.

Here, it is illustrated in the drawings that the driving part first jaw pulley is formed of two pulleys 211 and 212, the wire 301 is coupled to one pulley 211, and the wire 305 is coupled to the other pulley 212. However, an embodiment of the present disclosure is not limited thereto, and the driving part first jaw pulley may be formed of one pulley, and both the wires 301 and 305 may be coupled to the one pulley.

As described above, the driving part first jaw rotation shaft 241 is coupled to the first jaw driving motor (not shown) by the first jaw motor coupling part 251, and thus, when the first jaw driving motor (not shown) rotates for driving the first jaw 101, the pulleys 211 and 212, which are driving part first jaw pulleys, are rotated together with the driving part first jaw rotation shaft 241, so that the wires 301 and 305, which are first jaw wires, are pulled or released.

The pulleys 221 and 222, which are driving part second jaw rotation shafts, may be coupled to the driving part second jaw rotation shaft 242. Here, the pulley 221 and the pulley 222 may be formed to rotate together with the driving part second jaw rotation shaft 242.

In addition, the driving part second jaw auxiliary rotation shaft 246 may be disposed in a region adjacent to the driving part second jaw rotation shaft 242. The pulleys 223 and 224, which are driving part second jaw auxiliary pulleys, may be coupled to the driving part first jaw auxiliary rotation shaft 245. Here, the pulleys 223 and 224 may be formed to be rotatable around the driving part second jaw auxiliary rotation shaft 246.

Here, it is illustrated in the drawings that the driving part second jaw pulley is formed of two pulleys 221 and 222, the wire 302 is coupled to one pulley 221, and the wire 306 is coupled to the other pulley 222. However, an embodiment of the present disclosure is not limited thereto, and the driving part second jaw pulley may be formed of one pulley, and both the wires 302 and 306 may be coupled to the one pulley.

As described above, the driving part second jaw rotation shaft 242 is coupled to the second jaw driving motor (not shown) by the second jaw driving motor coupling part 252, and thus, when the second jaw driving motor (not shown) rotates for driving the second jaw 102, the pulley 221 and the pulley 222, which are driving part second jaw pulleys, are rotated together with the driving part second jaw rotation shaft 242, so that the wires 302 and 306, which are second jaw wires, are pulled or released.

The pulley 231, which is a driving part pitch pulley, may be coupled to the driving part pitch rotation shaft 243. Here, the pulley 231 may be formed to rotate together with the driving part pitch rotation shaft 243.

As described above, the driving part pitch rotation shaft 243 is coupled to a pitch driving motor (not shown) by the pitch driving motor coupling part 253, and thus, when the pitch driving motor (not shown) rotates for a pitch motion, the wires 303 and 304, which are pitch wires, are pulled or released as the pulley 231, which is a driving part pitch pulley, is rotated together with the driving part pitch rotation shaft 243.

Meanwhile, the pulley 215, the pulley 216, the pulley 217, the pulley 218, the pulley 225, the pulley 226, the pulley 227, and the pulley 228, which are driving part relay pulleys, may be formed to be rotatable around the driving part pitch rotation shaft 243 by inserting the driving part pitch rotation shaft 243 therethrough. Here, the pulley 215, the pulley 216, the pulley 217, and the pulley 218, which are driving part first jaw relay pulleys, may be disposed on one surface side of the pulley 231 that is a pitch pulley, and the pulley 225, the pulley 226, the pulley 227, and the pulley 228, which are driving part second jaw relay pulleys, may be disposed on the other surface side of the pulley 231.

Viewed from another perspective, along the driving part pitch rotation shaft 243, the pulleys 225 and 226, which are driving part second jaw first relay pulleys, the pulleys 227 and 228, which are driving part second jaw second relay pulleys, the pulley 231, which is a driving part pitch pulley, and the pulleys 217 and 218, which are driving part first jaw second relay pulleys, and the pulleys 215 and 216, which are driving part first jaw first relay pulleys, are sequentially stacked and formed.

In addition, the pitch-yaw connector 232 may be coupled to the driving part pitch rotation shaft 243. The pitch-yaw connector 232 may be formed to rigidly connect the pulley 231, which is a driving part pitch pulley, to the pulley 219, the pulley 220, the pulley 229, and the pulley 230, which are driving part satellite pulleys to allow the driving part satellite pulleys to be revolved around the driving part pitch rotation shaft 243 when the pulley 231 is rotated. This will be described in detail later.

Here, the pitch-yaw connector 232 may be formed to rotate together with the driving part pitch rotation shaft 243. That is, the pulley 231 and the pitch-yaw connector 232 may be coupled to the driving part pitch rotation shaft 243, and may be rotated together with the driving part pitch rotation shaft 243.

Here, the pitch-yaw connector 232 may be described as being formed in an approximately Y-shape as shown in FIG. 12, or the pitch-yaw connector 232 may be described as being formed in a shape in which at least two extension portions 232a and 232b are formed to extend from the center thereof. In addition, a driving part first jaw satellite pulley central shaft 233 and a driving part second jaw satellite pulley central shaft 234 may be formed at end portions of the extension portions 232a and 232b, respectively.

In addition, the pulleys 219 and 220, which are driving part first jaw satellite pulleys, may be coupled to the driving part first jaw satellite pulley central shaft 233, and the pulleys 229 and 230, which are driving part second jaw satellite pulleys, may be coupled to the driving part second jaw satellite pulley central shaft 234.

As a result, when the pulley 231, which is a driving part pitch pulley, is rotated together with the driving part pitch rotation shaft 243, the pulley 219, the pulley 220, the pulley 229, and the pulley 230, which are driving part satellite pulleys, are revolved around the driving part pitch rotation shaft 243. In other words, it may be said that the driving part first jaw satellite pulley central shaft 233 and the driving part second jaw satellite pulley central shaft 234 are rotated around the driving part pitch rotation shaft 243 while maintaining a constant distance from the driving part pitch rotation shaft 243 in a state in which the driving part first jaw satellite pulley central shaft 233 and the driving part second jaw satellite pulley central shaft 234 are spaced apart from the driving part pitch rotation shaft 243 by a certain extent.

That is, the driving part satellite pulley is formed to be movable relative to the driving part relay pulley and the driving part pitch rotation shaft 243 so that a relative position of the driving part satellite pulley with respect to the driving part relay pulley and the driving part pitch rotation shaft 243 may be changed. On the other hand, the relative positions of the driving part pitch pulley 231 and the driving part relay pulley remain constant.

In addition, when the pulley 231, which is a driving part pitch pulley, is rotated around the driving part pitch rotation shaft 243, the pulley 219, the pulley 220, the pulley 229, and the pulley 230, which are driving part satellite pulleys, are moved relative to the pulley 231, which is a driving part pitch pulley, so that the overall lengths of the wire 301, the wire 302, the wire 305, and the wire 306, which are jaw wires, in the driving part 200 are changed.

The wire 301, which is a first jaw wire, is connected to the end tool 100 through the connection part 310 after being sequentially wound to make contact with at least portions of the pulley 211, the pulley 213, the pulley 215, the pulley 219, and the pulley 217 in a state in which one end portion of the wire 301 is coupled to the pulley 211 by the first jaw wire-driving part coupling member (not shown).

Viewed from another perspective, the wire 301, which is a first jaw wire, is connected to the end tool 100 through the connection part 310 after being sequentially passing through the driving part first jaw pulley 211, the driving part first jaw auxiliary pulley 213, the driving part first jaw first relay pulley 215, the driving part first jaw satellite pulley 219, and the driving part first jaw second relay pulley 217.

Viewed from another perspective, the wire 301, which is a first jaw wire, enters the driving part 200 after passing through the end tool 100 and the connection part 310, and then is fixedly coupled to the pulley 211, which is a driving part first jaw pulley after being sequentially wound around the pulley 217, the pulley 219, the pulley 215, and the pulley 213.

Meanwhile, the wire 305, which is a first jaw wire, is connected to the end tool 100 through the connection part 310 after being sequentially wound to make contact with at least portions of the pulley 212, the pulley 214, the pulley 216, the pulley 220, and the pulley 218 in a state in which one end portion of the wire 305 is coupled to the pulley 212 by the first jaw wire-driving part coupling member (not shown).

The wire 302, which is a second jaw wire, is connected to the end tool 100 through the connection part 310 after being sequentially wound to make contact with at least portions of the pulley 221, the pulley 223, the pulley 225, the pulley 229, and the pulley 227 in a state in which one end portion thereof is coupled to the pulley 221 by the second jaw wire-driving part coupling member (not shown).

Meanwhile, the wire 306, which is a second jaw wire, is connected to the end tool 100 through the connection part 310 after being sequentially wound to make contact with at least portions of the pulley 222, the pulley 224, the pulley 226, the pulley 230, and the pulley 228 in a state in which one end portion thereof is coupled to the pulley 222 by the second jaw wire-driving part coupling member (not shown).

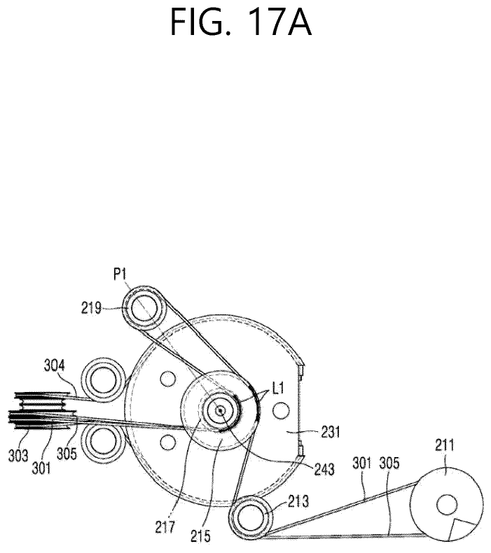

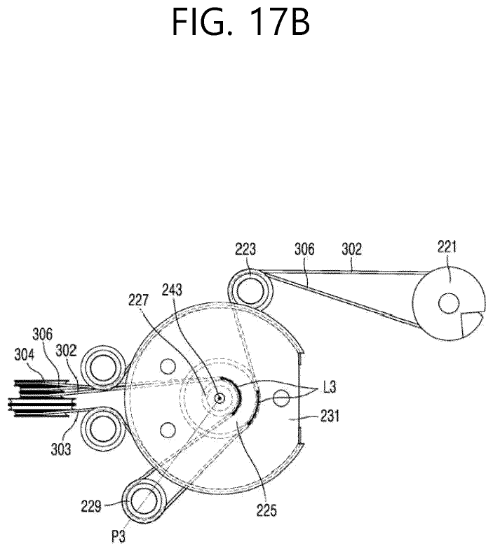

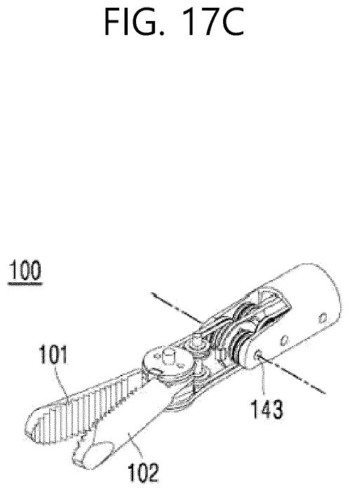

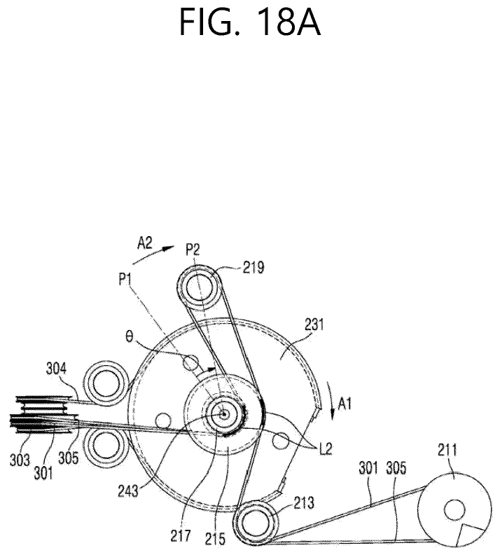

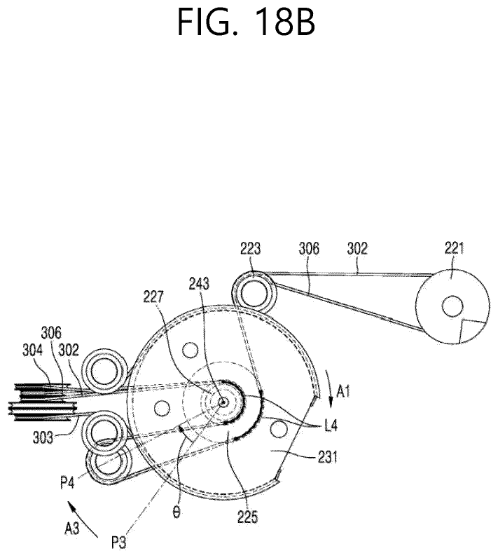

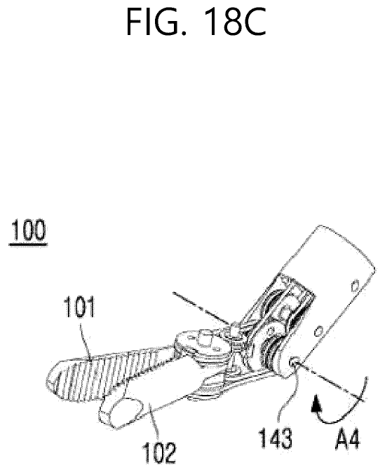

FIGS. 17 and 18 are views illustrating a pitch motion of the multi-joint type surgical instrument illustrated in FIG. 6. Here, for convenience of description, only the pulleys and wires related to the rotation of the first jaw are illustrated in FIG. 17A and FIG. 18A, and only the pulleys and wires related to the rotation of the second jaw are illustrated in FIG. 17B and FIG. 18B. In addition, FIG. 17C and FIG. 18C illustrate a pitch motion of the end tool according to a pitch motion of the driving part.

Here, in the multi-joint type surgical instrument 30 according to an embodiment of the present disclosure, when the driving part satellite pulley is moved relative to the driving part relay pulley, which causes the overall length of the jaw wire to be changed in the driving part 200, allowing the end tool 100 to perform a pitch motion. In particular, in the multi-joint type surgical instrument 30 according to an embodiment of the present disclosure, when the driving part pitch pulley 231 is rotated, which causes the driving part satellite pulley to be revolved around the (common) rotation shaft of the driving part relay pulley and the driving part pitch pulley 231 so that a path length of the jaw wire wound around the driving part relay pulley is changed, allowing the end tool to perform a pitch motion.

Specifically, when a motion compensation for the pitch motion is not separately performed in the driving part, the pitch motion itself cannot be performed in the end tool.

Meanwhile, in order for the end tool to perform a pitch motion, the wires 301 and 305 should be further wound around the pulley 113 by ΔSpitch and the wires 302 and 306 should be further unwound from the pulley 114 by ΔSpitch. However, when such compensation is not performed in the driving part, the pitch motion itself cannot be performed in the end tool.

In order to perform motion compensation for the pitch motion as described above, in the multi-joint type surgical instrument 30 according to an embodiment of the present disclosure, the driving part pitch pulleys are rotated while the driving part satellite pulleys are revolved, so that the jaw wires are wound around or released from the driving part relay pulley, which allows the movement of the jaw wires to be compensated for by the rotation of the driving part pitch pulley 231.

In other words, when the pulley 231, which is a driving part pitch pulley, is rotated together with the driving part pitch rotation shaft 243, the driving part satellite pulleys are revolved around the driving part pitch rotation shaft 243. In addition, as the driving part satellite pulleys are revolved around the driving part pitch rotation shaft 243, the jaw wire wound around the driving part relay pulley is changed in length. That is, the jaw wire wound at the end tool 100 side due to the rotation of the pulley 231 is released by the same amount at the driving part 200 side, and the jaw wire unwound at the end tool 100 side is wound by the same amount at the driving part 200 side, so that the pitch motion does not affect the yaw motion.

Viewed from another perspective, when the end tool performs a pitch motion due to the rotation of the driving part pitch pulley 231, the jaw wire (responsible for the yaw and actuation motions) is also moved by the pitch motion. That is, as the pitch rotation is performed around the rotation shaft 143 of the end tool 100, both strands of the jaw wire coupled to one jaw are pulled, and both strands thereof coupled to the other jaw are released. Accordingly, it may be described that in the present disclosure, in order to compensate for the movement of the jaw wire, when the end tool performs the pitch motion, the overall length of the jaw wire in the driving part is changed while the driving part satellite pulley is moved relative to the driving part relay pulley, so that the jaw wire is released (or pulled) at the end tool side as much as the jaw wire is pulled (or released) at the driving part side, thereby compensating for the movement of the jaw wire when the end tool performs the pitch motion.

Hereinafter, the pitch motion will be described in more detail.

When the pulley 231, which is a driving part pitch pulley, is rotated in the direction of an arrow A1 (i.e., in the clockwise direction in the drawing) in order for the pitch motion, the pitch-yaw connector 232 (see FIG. 10) is rotated in the direction of the arrow A1 together with the pulley 231, and thus, the pulleys 219 and 220, which are driving part satellite pulleys fixedly coupled to the pitch-yaw connector 232 (see FIG. 10), are revolved as a whole in the direction of an arrow A2 of FIG. 18A (i.e., in the clockwise direction in the drawing) around the driving part pitch rotation shaft 243 by θ. That is, when the pulley 231 is rotated, the pulleys 219 and 220 are revolved by θ from the position of P1 of FIG. 17A to the position of P2 of FIG. 18A. Viewed from another perspective, it may be described that when the driving part pitch pulley 231 is rotated, the driving part satellite pulley is moved in conjunction with the driving part pitch pulley 231.

At the same time, when the pulley 231, which is a driving part pitch pulley, is rotated in the direction of the arrow A1 (i.e., in the clockwise direction in the drawing), the pitch-yaw connector 232 (see FIG. 10) is rotated in the direction of the arrow A1 together with the pulley 231, and thus, the pulleys 229 and 230, which are driving part satellite pulleys fixedly coupled to the pitch-yaw connector 232 (see FIG. 10), are revolved as a whole in the direction of an arrow A3 of FIG. 18B (i.e., in the clockwise direction in the drawing) around the driving part pitch rotation shaft 243 by θ. That is, when the pulley 231 is rotated, the pulleys 229 and 230 are revolved by θ from the position of P3 of FIG. 17B to the position of P4 of FIG. 18B. Viewed from another perspective, it may be described that when the driving part pitch pulley 231 is rotated, the driving part satellite pulley is moved in conjunction with the driving part pitch pulley 231.

Meanwhile, in this case, the positions of the pulley 215, the pulley 216, the pulley 217, the pulley 218, the pulley 225, the pulley 226, the pulley 227, and the pulley 228, which are driving part relay pulleys coupled to the driving part pitch rotation shaft 243, are not changed. That is, the relative positions of the pulley 211, which is a driving part jaw pulley, the pulley 231, which is a driving part pitch pulley, and the pulley 215, the pulley 216, the pulley 217, and the pulley 218, which are driving part relay pulleys, remain constant. Similarly, the relative positions of the pulley 221, which is a driving part jaw pulley, the pulley 231, which is a driving part pitch pulley, and the pulley 225, the pulley 226, the pulley 227, and the pulley 228, which are driving part relay pulleys, remain constant.

In addition, as described above, the relative position of the driving part satellite pulley with respect to the driving part relay pulley is changed as the driving part satellite pulley is revolved, and thus, the length of each wire wound around the driving part relay pulley, that is, the path length, is changed. Here, since the driving part relay pulley includes the pulley 215, which is a driving part first jaw first relay pulley, and the pulley 217, which is a driving part first jaw second relay pulley, the path length also means the sum of the length of the wire 301 wound around the pulley 215 and the length of the wire 301 wound around the pulley 217 (or, the sum of the length by which the wire 305 is wound around the pulley 216 and the length by which the wire 305 is wound on the pulley 218).

That is, as compared to a path length L1 by which the wires 301 and 305, which are first jaw wires, wound around the driving part relay pulleys at the position of (a) of FIG. 17, a path length L2 by which the first jaw wires wound around the driving part relay pulleys at the position of FIG. 18A is reduced, and thus, the first jaw wires are further released at the driving part 200 side by the reduced path length (L1-L2). That is, the overall lengths of the wires 301 and 305, which are first jaw wires, in the driving part 200 are reduced. In addition, as the overall length of the first jaw wire in the driving part 200 is reduced, the overall length of the first jaw wire in the end tool 100 is increased as much as the first jaw wire is unwound.

In contrast, when the pulley 231, which is a driving part pitch pulley, is rotated in the direction of the arrow A1, as compared to a path length L3 by which the wires 302 and 306, which are second jaw wires, wound around the driving part relay pulleys at the position of FIG. 17B, a path length L4 by which the second jaw wires wound around the driving part relay pulleys at the position of FIG. 18B is increased, and the second jaw wires are further pulled at the driving part 200 side by as much as the increased path length (L4-L3). That is, the overall lengths of the wires 302 and 306, which are second jaw wires, in the driving part 200 are increased. In addition, as the overall length of the second jaw wire in the driving part 200 is increased, the overall length of the second jaw wire in the end tool 100 is reduced as much as the second jaw wire is pulled.

As such, when the pulley 231, which is a driving part pitch pulley, is rotated in the direction of the arrow A1 for a pitch motion, the relative position of the driving part satellite pulley is changed as the driving part satellite pulley is moved relative to the driving part pitch pulley 231 and the driving part relay pulley. In addition, due to the relative movement of the driving part satellite pulley, the overall length of the first jaw wire in the driving part 200 is reduced, and the overall length of the first jaw wire in the end tool 100 is increased. At the same time, due to the relative movement of the driving part satellite pulley, the overall length of the second jaw wire in the driving part 200 is increased, and the overall length of the second jaw wire in the end tool 100 is reduced.

As a result, when the pulley 231, which is a driving part pitch pulley, is rotated in the direction of the arrow A1, the wires 301 and 305, which are two strands of the first jaw wire, are released and the wires 302 and 306, which are two strands of the second jaw wire, are pulled when viewed from the end tool 100 side, so that the end tool 100 performs a pitch motion in the direction of an arrow A4 around the rotation shaft 143.

Here, the term “path length” may be defined as a length of the jaw wire from a point at which the jaw wire enters the driving part first relay pulley to a point at which the jaw wire exits from the driving part second relay pulley through the driving part satellite pulley. That is, the path length may be defined as a length of the wire 301, which is a jaw wire, from a point at which the jaw wire enters the pulley 215, which is a driving part first relay pulley, to a point at which the jaw wire exits from the pulley 217, which is a driving part second relay pulley, through the pulley 219 that is a driving part satellite pulley.

Viewed from another perspective, the path length may be defined as the length of the jaw wire from an initial contact point of the jaw wire with the driving part relay pulley to a final contact point of the jaw wire with the driving part relay pulley on a deployment path of the jaw wire that connects the end tool jaw pulley to the driving part jaw pulley. That is, the path length may be defined as the length of the jaw wire from an initial contact point of the wire 301, which is a jaw wire, with the pulley 215, which is a driving part first relay pulley, to a final contact point of the wire 301 with the pulley 217, which is a driving part second relay pulley.

Meanwhile, as the above-described path length is changed while the driving part satellite pulley is moved relative to the driving part relay pulley, the overall length of the jaw wire in the driving part 200 is also changed. In addition, as the overall length of the jaw wire in the driving part 200 is changed, the overall length of the jaw wire in the end tool 100 is also changed. However, it may be said that since the overall length of the jaw wire in the end tool 100 is also increased (or reduced) by as much as the overall length of the jaw wire increased (reduced) in the driving part 200, a total length of the jaw wire is not changed (assuming that elastic deformation or the like is not considered).

As a result, when the driving part pitch pulley 231 is rotated, the wire 301/wire 305, which are first jaw wires, are released at the driving part 200 side by as much as the wire 301/wire 305, which are first jaw wires, are pulled at the end tool 100 side, as a result, a pitch motion is enabled.

Meanwhile, as described above, the end tool 100 of the multi-joint type surgical instrument 30 of the present disclosure may further include the pulley 131, which is an end tool pitch pulley, the driving part 200 may further include the pulley 231, which is a driving part pitch pulley, and the power transmission part 300 may further include the wire 303 and the wire 304 which are pitch wires.

Accordingly, when the pulley 231, which is a driving part pitch pulley, is rotated in the direction of the arrow A1, due to the rotation of the pulley 231, the wire 304 is wound around the pulley 231 and the wire 303 is released from the pulley 231. Accordingly, the pulley 131, which is an end tool pitch pulley connected to the other sides of the wires 303 and 304, is rotated in the direction of the arrow A2 around the rotation shaft 143, so that the pitch motion may be more surely and reliably performed.

Here, among the pulleys that are rotated around the rotation shaft 143, which is an end tool pitch rotation shaft, the pulley 131, which is an end tool pitch pulley in contact with the wires 303 and 304 that are pitch wires, may be formed to have a diameter different from those of the pulley 113, the pulley 114, the pulley 123, and the pulley 124, which are end tool jaw pitch main pulleys in contact with the wire 301, the wire 305, the wire 302, and the wire 306 that are jaw wires.

In this case, when the rotation shaft 143 is rotated, the lengths of the wires wound around or unwound from the respective pulleys are different from each other. For example, when a diameter of the end tool pitch pulley is 6 φ, a diameter of the end tool jaw pitch main pulley is 4 φ, and the rotation shaft 143 is rotated by 90°, a length of the pitch wire wound around the end tool pitch pulley is 1.5 π, whereas a length of the jaw wire wound around the end tool jaw pitch main pulley may be 1 π.

From this perspective, the length of the wire wound around or unwound from the pulley may be defined as “rotation amount”. The rotation amount is a concept different from a rotation angle, and may be calculated as (diameter*rotation angle/360°*π).

In this case, since essentially the pulley 231, which is a driving part pitch pulley, is directly connected to the pulley 131, which is an end tool pitch pulley, by the wires 303 and 304, which are pitch wires, the rotation amount of the driving part pitch pulley 231 is the same as that of the end tool pitch pulley. That is, the pitch wire is released from or wound around the end tool pitch pulley by as much as the pitch wire is wound around or released from the driving part pitch pulley 231.

Meanwhile, a relation of (diameter of end tool pitch pulley:diameter of end tool jaw pitch main pulley)=(rotation amount of wire wound around end tool pitch pulley:rotation amount of wire wound around end tool jaw pitch main pulley) may be established.

As described above, when, in the end tool 100, the length of the pitch wire wound around the end tool pitch pulley is different from the length of the jaw wire wound around the end tool jaw pitch main pulley, in the driving part 200, the length of the pitch wire to be released should be different from the length of the jaw wire to be released by the same proportion. To this end, the relationship of (diameter of end tool pitch pulley:diameter of end tool jaw pitch main pulley)=(diameter of driving part pitch pulley:diameter of driving part relay pulley) may be established.

For example, when a ratio of (diameter of end tool pitch pulley:diameter of end tool jaw pitch main pulley) is 6:4, a ratio of (diameter of driving part pitch pulley:diameter of driving part relay pulley) may also be 6:4. According to this ratio, the diameter of the driving part pitch pulley may be 9 φ, and the diameter of the driving part relay pulley may be 6 φ.

However, here, the driving part relay pulley may include two or more pulleys including the driving part first relay pulley and the driving part second relay pulley. In addition, the sum of the diameters of the driving part first relay pulley and the driving part second relay pulley may be defined as the diameter of the driving part relay pulley.

For example, when the diameter of the driving part relay pulley is 6φ, there are several possible combinations for (diameter of driving part first relay pulley, diameter of driving part second relay pulley), including (1φ, 5φ), (2φ, 4φ), (3φ, 3φ), (4φ, 2φ), and (5φ, 1φ), among others. Here, it is illustrated in the drawings that the diameter of the pulley 215, which is a driving part first relay pulley, is 4 φ, and the diameter of the pulley 217, which is the driving part second relay pulley, is 2 φ.

In addition, it may be described that rotation amount of driving part first relay pulley plus the rotation amount of driving part second relay pulley is proportional to the rotation amount of the driving part pitch pulley.

However, although the ratio of (diameter of end tool pitch pulley:diameter of end tool jaw pitch main pulley) may not exactly match the ratio of (diameter of driving part pitch pulley:diameter of driving part relay pulley), when the pulley diameters are selected to make these ratios similar, the object of the present disclosure, which is to compensate for the movement of the jaw wire with the rotation of the driving part pitch pulley, can be achieved to some extent.

The process of the final pitch motion will be described again as follows.

Hereinafter, a case in which the diameter of the end tool pitch pulley is 6 φ, the diameter of the end tool jaw pitch main pulley is 4 φ, the diameter of the driving part pitch pulley is 9 φ, and the diameter of the driving part relay pulley is 6 φ will be described as an example.

First, for a pitch motion, the pulley 231, which is a driving part pitch pulley of the driving part 200, is rotated by 60° to wind the wire 304, which is a pitch wire, while releasing the wire 303. At this time, the length of the wire 303/wire 304 wound and unwound is 1.5 π.

Accordingly, as the wire 304 is pulled by 1.5 π and the wire 303 is released by 1.5 π in the end tool 100, the pulley 131, which is an end tool pitch pulley, is rotated by 90° corresponding to 1.5 π.

Meanwhile, when the pulley 131 is pitch-rotated around the rotation shaft 143, the jaws 101 and 102 and the pulley 111/pulley 112 are also pitch-rotated around the rotation shaft 143. Accordingly, the wires 301 and 305, which are first jaw wires coupled to the pulley 111, are both pulled, and the wires 302 and 306, which are second jaw wires coupled to the pulley 121, are both released. At this time, the angles by which the end tool pitch pulley and the end tool jaw pitch main pulley are rotated are equal to each other and measure 90°, and thus, the length of the jaw wires wound around or released from the end tool jaw pitch main pulley becomes 1 π.

Meanwhile, since the pulley 231 and the pulley 219/pulley 220 are rigidly connected by the pitch-yaw connector 232, when the pulley 231 is rotated by 60° around the driving part pitch rotation shaft 243, the pulley 219/pulley 220 are revolved by 60° around the driving part pitch rotation shaft 243.

In addition, as described above, as the pulley 219/pulley 220 are revolved, the jaw wires are wound around or released from the pulley 215 and the pulley 216, whose combined diameter is 6 φ, by 1 π corresponding to a revolution angle of 60°. That is, the wires 301 and 305, which are first jaw wires, are released as a whole, and the wires 302 and 306, which are second jaw wires, are pulled as a whole.

In other words, the overall path lengths of the wires 301 and 305 wound around the pulley 215, the pulley 216, the pulley 217, and the pulley 218, which are driving part first jaw relay pulleys, are reduced, and the wires 301 and 305 are released by as much as the reduced path length. In addition, the overall path lengths of the wires 302 and 306 wound around the pulley 225, the pulley 226, the pulley 227, and the pulley 228, which are driving part second jaw relay pulleys, are increased, and the wires 302 and 306 are pulled by as much as the increased path length.

That is, the wires 301 and 305, which are first jaw wires, are released at the driving part 200 side by as much as the wires 301 and 305 are pulled at the end tool 100 side, thereby compensating for the movement of the jaw wire due to the pitch motion. Similarly, the wires 302 and 306, which are second jaw wires, are released at the driving part 200 side by as much as the wires 302 and 306 are pulled at the end tool 100 side, thereby compensating for the movement of the jaw wire due to the pitch motion.

As a result, by releasing (or pulling) the jaw wires at the driving part 200 side by as much as a length equal to the length by which the jaw wires are wound around (or released from) the end tool 100 side in response to the pitch motion, the pitch motion can be performed independently without affecting the rotation of the jaw around the yaw shaft.

That is, when the driving part pitch pulley 231 and the driving part satellite pulley are rigidly connected, and the driving part pitch pulley 231 is rotated around the driving part pitch rotation shaft 243, the path length of the jaw wire wound around the driving part relay pulley is changed as the driving part satellite pulley is revolved around the driving part pitch rotation shaft 243. In addition, the change in the path length of the jaw wire compensates for the movement of the jaw wires at the end tool side due to the pitch motion, as a result, the pitch motion is independently performed.

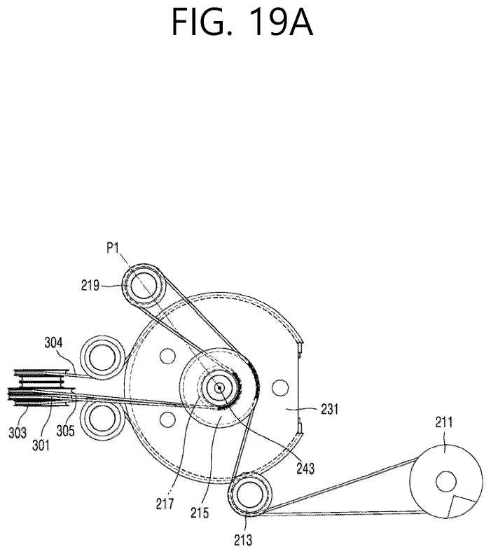

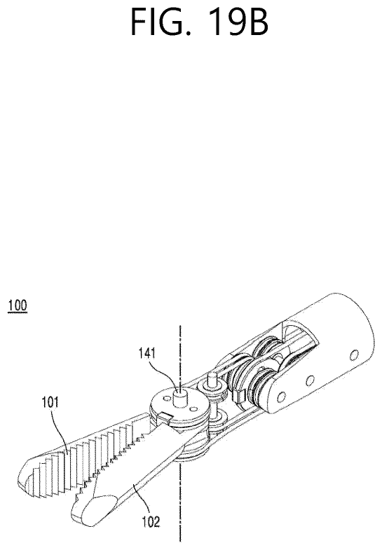

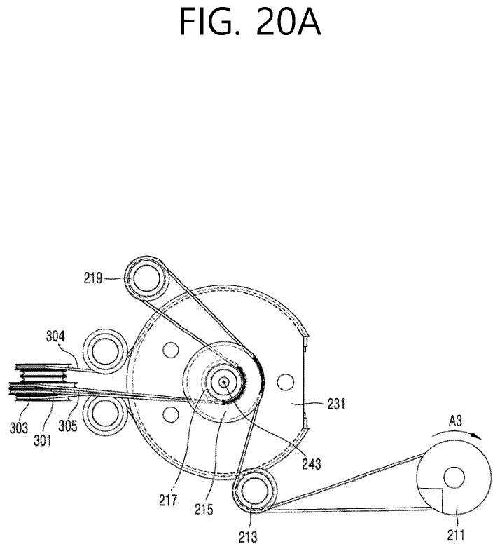

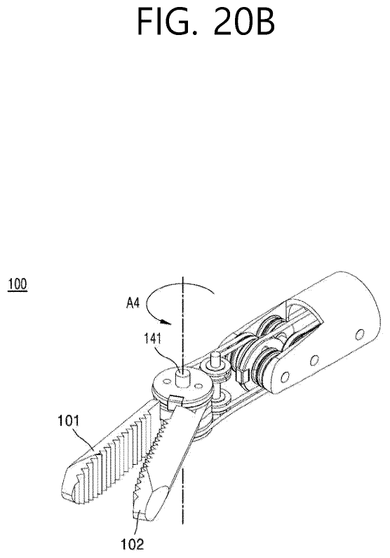

FIGS. 19A to 20B are views illustrating a yaw motion of the multi-joint type surgical instrument illustrated in FIG. 6.

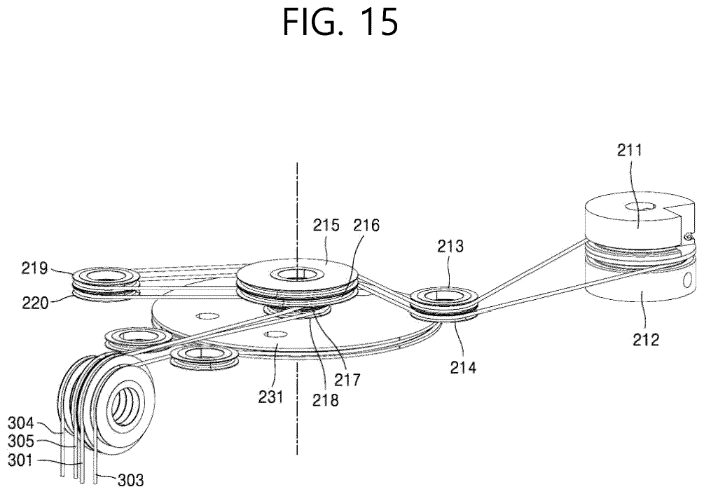

Referring to FIGS. 15, 16, 19, and 20 and the like, when the pulley 211, which is a driving part first jaw pulley, is rotated in the direction of an arrow A3 for a yaw motion, one of the wires 301 and 305, which are first jaw wires, is wound around the pulley 211 and the other one thereof is released from the pulley 211 in response to the rotation of the pulley 211. Accordingly, the pulley 111, which is an end tool first jaw pulley connected to the opposite side of the wires 301 and 305, is rotated in the direction of as arrow A4, so that the yaw motion is performed.

At this time, the pulley 219, the pulley 220, the pulley 229, and the pulley 230, which are driving part satellite pulleys, and the pulley 215, the pulley 216, the pulley 217, the pulley 218, the pulley 225, the pulley 226, the pulley 227, and the pulley 228, which are driving part relay pulleys, are not changed in position, but only the motion in which the wires 301 and 305 are wound around or released from the driving part satellite pulley and the driving part relay pulley occurs.

Accordingly, the driving part pitch pulley 231 rigidly connected to the driving part satellite pulley is not rotated, and the wires 303 and 304, which are pitch wires, are not wound or released and maintained in position.

Similarly, when the pulley 221, which is a driving part second jaw pulley, is rotated for a yaw motion, in response to the rotation of the pulley 221, one of the wires 302 and 306, which are second jaw wires, is wound around the pulley 221 and the other one thereof is released from the pulley 221. Accordingly, the pulley 121, which is an end tool second jaw pulley connected to the opposite side of the wires 302 and 306, is rotated in one direction, so that the yaw motion is performed.

At this time, the pulley 219, the pulley 220, the pulley 229, and the pulley 230, which are driving part satellite pulleys, and the pulley 215, the pulley 216, the pulley 217, the pulley 218, the pulley 225, the pulley 226, the pulley 227, and the pulley 228, which are driving part relay pulleys, are not changed in position, but only the motion in which the wires 302 and 306 are wound around or released from the driving part satellite pulley and the driving part relay pulley occurs.

Accordingly, the driving part pitch pulley 231 rigidly connected to the driving part satellite pulley is not rotated, and the wires 303 and 304, which are pitch wires, are not wound or released and maintained in position.

As a result, the overall lengths of the wire 301, the wire 302, the wire 305, and the wire 306, which are jaw wires, in the driving part 200 remain constant even when the pulley 211 or pulley 221, which is a driving part jaw pulley, is rotated for the yaw or actuation motion.

As described above, in the multi-joint type surgical instrument 30 according to an embodiment of the present disclosure, when the driving part pitch pulley 231 is rotated, the driving part satellite pulley is revolved around the rotation shaft of the driving part pitch pulley 231 to change the path length of the jaw wire wound around the driving part relay pulley, and the jaw wire is wound or released in response to the rotation of the driving part pitch pulley 231, so that the movement of the jaw wire due to the pitch drive may be offset or compensated, and as a result, the effect of separating the pitch motion and the yaw motion can be obtained.

However, the pitch motion and the yaw motion are not limited to being mechanically separated from each other as described above, and can be separated and performed independently by the processor according to an embodiment of the present disclosure.

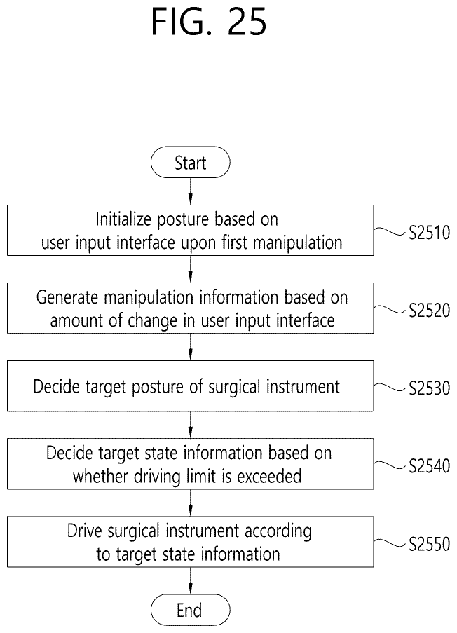

FIG. 25 is a schematic flowchart of a method for driving a surgical instrument according to an embodiment of the present disclosure.

Referring to FIG. 25, a method for driving a surgical instrument may be configured of stages processed in time series in the user terminals 2000 and 2010 or the processor 2011 illustrated in FIGS. 1 and 2A. Accordingly, even when the content is omitted hereinafter, the content described above regarding the user terminals 2000 and 2010 or the processor 2011 illustrated in FIGS. 1 and 2A may also be applied to the method for driving the surgical instrument of FIG. 25.

In addition, as described above with reference to FIGS. 1 and 2B, at least one of the stages of the method for deriving the surgical instrument of FIG. 25 may be processed by the servers 3000 or the processor 3011.

In addition, as described above with reference to FIGS. 3 to 5, at least one of the stages of the method for driving the surgical instrument of FIG. 25 may be processed by the master robot 10, the slave robot 20, the multi-joint surgical instrument 30, or a processor included therein.

Hereinafter, for convenience of explanation, the method for driving the surgical instrument according to embodiments of the present disclosure may be described as being performed by a computing device. The computing device may be, for example, the aforementioned user terminal, server, master robot, slave robot, multi-joint surgical instrument, a processor included therein, or a combination thereof, but is not limited thereto. Those skilled in the art will easily understand that any apparatus capable of arbitrary calculation including a processor and memory may perform the method for driving the surgical instrument according to embodiments of the present disclosure as a computing apparatus.

To drive the surgical instrument, the computing device first initializes the reference posture information (To c) of the user input interface when starting to control the surgical instrument (stage 2510). In other words, the reference posture of the user input interface may be updated with posture information prior to the manipulation of the user input interface before the first manipulation of the user input interface. According to an aspect, control of the surgical instrument may be performed based on the degree to which the user input interface has changed, so the reference posture of the user input interface, which serves as a reference for determining the degree of change, is initialized prior to manipulation by a user. Herein, the user input interface may be, for example, a manipulation lever provided in the master robot, but is not limited thereto.

Herein, the posture information may include position and orientation information on a three-dimensional coordinate system, and may be, as a non-limiting example, expressed in the form of a Homogeneous transform matrix (T), which is a 4×4 matrix as shown in Equation 1 below, without being limited thereto.

$$T = [ \begin{matrix}{R}_{11} & {R}_{12} & {R}_{13} & {p}_{1} \\ {R}_{21} & {R}_{22} & {R}_{23} & {p}_{2} \\ {R}_{31} & {R}_{32} & {R}_{33} & {p}_{3} \\ 0 & 0 & 0 & 1\end{matrix} ] = [ \begin{matrix}R & p \\ 0 & 1\end{matrix} ] \qquad [ Equation 1 ]$$

The homogeneous transform matrix physically refers to a change in position and/or orientation from the reference coordinate system defined in the user input interface to the current posture coordinate system of the user input interface. Here, the posture information does not necessarily need to be expressed in the form of a homogeneous transform matrix, and the use of any expression method such as a screw method, for example, should also be understood as being included in the technical spirit of the present disclosure.

As illustrated in FIG. 25, in order to perform the method for driving the surgical instrument according to an embodiment of the present disclosure, the computing device may generate manipulation information based on an amount of change in the reference posture of the user input interface for controlling the surgical instrument (stage 2520). In other words, manipulation information may be generated by a user manipulating the user input interface. The manipulation information (T^{MC}) for the user input interface may include an amount of change from the reference posture information (T₀^{MC}) of the user input interface to the posture information (T_{curr}^{MC}) of the user input interface created by manipulation by a user. To this end, for example, an inverse matrix and multiplication calculation may be used as shown in Equation 2 below.

$${T}^{MC} = {( {T}_{0}^{MC} )}^{- 1} {T}_{curr}^{MC} \qquad [ Equation 2 ]$$

Herein, the reference posture information of the user input interface may include information on the posture itself at a specific point in time of the user input interface, and the manipulation information of the user input interface may include information on an amount of change from the reference posture of the user input interface. The reference posture information and manipulation information may be expressed in the same form, for example, as a homogeneous transform matrix. According to an aspect, the reference posture information may be understood as representing the degree of change from the origin of the coordinate system, thereby representing information on the reference posture of the user input interface, and the manipulation information may be understood as representing the degree of change from the reference posture.

According to an embodiment, the generated posture information of the user input interface may be transmitted to the surgical instrument. According to another aspect, information on the target posture generated based on posture information, which will be described later, or information on the target state of the driving element may be transmitted to the surgical instrument. In other words, the computing device that performs the method for driving the surgical instrument according to an embodiment of the present disclosure may be a separate apparatus separate from the surgical robot or the surgical instrument, for example, an apparatus including a processor of the master robot. Alternatively, it may be understood as an apparatus that includes both the processor of the master robot and the processor of the surgical robot.

Referring again to FIG. 25, the computing device may decide the target posture of the surgical instrument corresponding to the previously obtained manipulation information of the user input interface (stage 2530). According to an aspect, the computing device may be configured to decide the target posture of the surgical instrument based on the correspondence relationship between the predetermined movement of the user input interface and the movement of the surgical instrument.

As a non-limiting example, the computing device may generate the target posture (T^{SR}) of the surgical instrument based on at least one of the manipulation information of the user input interface (T^{MC}), a rotation matrix that transforms the reference coordinate system of the user input interface to the reference coordinate system of the surgical instrument (R_{MC→SR}), a rotation matrix that transforms the reference coordinate system of the user input interface to the reference posture coordinate system of the surgical instrument (R_{MC→SR0}), a rotation matrix that transforms the reference coordinate system of the surgical instrument to a camera viewing coordinate system (R_{SR→cam}), or a current posture of the surgical instrument (T₀^{SR}), as illustrated in Equation 3 below.

$${T}^{SR} = {f}_{1} ( {T}^{MC} , {R}_{MC \to SR} , {R}_{MC \to SR 0} , {R}_{SR \to cam} , {T}_{0}^{SR} ) \qquad [ Equation 3 ]$$

In other words, the computing device may use a rotation matrix (R_{MC→SR}) to transform the reference coordinate system of the user input interface to the reference coordinate system of the surgical instrument, and transfer the manipulation information expressed by the reference coordinate system of the user input interface to the reference coordinate system of the surgical instrument. In addition, the computing device may reflect information on the reference posture of the surgical instrument using a rotation matrix (R_{MC→SR0}) that transforms the reference coordinate system of the user input interface to the reference posture coordinate system of the surgical instrument. In addition, the computing device may use a rotation matrix (R_{SR→cam}) that transforms the reference coordinate system of the surgical instrument to the camera viewing coordinate system to reflect the operating state of the surgical instrument and the state in which a camera is capturing, thereby allowing a user to more intuitively understand the driving state of the surgical instrument. In addition, the computing device may decide a reference for the degree of manipulation by manipulation information based on information on the current posture of the surgical instrument (T₀^{SR}) and decide the target posture of the surgical instrument accordingly.

However, the decision of the target posture of the surgical instrument corresponding to the manipulation information of the user input interface may be freely changed depending on the embodiment or the settings of a user. For example, the degree to which the surgical instrument is driven in response to an amount of change in the user input interface may be set differently depending on whether a user wants more detailed control or immediate control. In the method for driving the surgical instrument according to an embodiment of the present disclosure, the target posture corresponding to the manipulation information should be understood to include all various embodiments decided according to a predetermined correspondence relationship.

Referring again to FIG. 25, the computing device may decide the target state information for the driving element based on whether the decided target posture of the surgical instrument causes the driving limit for the at least one driving element provided in the surgical instrument to be exceeded (stage 2540). Herein, according to an aspect, the at least one driving element provided in the surgical instrument may include a joint, and in this connection, the target state information may be a target joint angle. In addition, the driving limit for the driving element may be represented by a joint limit angle. However, the technical idea of the present disclosure is not limited only to driving of a joint. It should be understood that it may be applied to driving elements of any surgical instrument, including bending-type driving elements, sliding-type driving elements, and rotating-type driving elements. Hereinafter, for convenience of explanation, the case where the driving element is a joint may be mainly described as an example. In other words, according to a non-limiting example, the computing device may decide whether at least one joint provided in the surgical instrument needs to exceed the joint limit angle in order to achieve the target posture of the surgical instrument, and decide the target joint angle for at least one joint provided in the surgical instrument depending on whether the joint limit angle is exceeded.

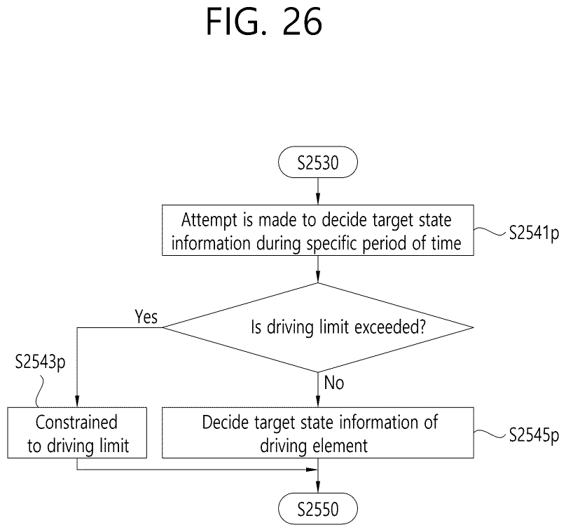

FIG. 26 shows a conceptual information processing procedure for the target state information decision stage of FIG. 25. More specifically, according to an aspect, the computing device may decide modified state information in which the driving result of the driving element is constrained to a range within the driving limit as the target state information of the at least one driving element provided in the surgical instrument in response to a decision that the decided target posture exceeds a driving limit for the at least one driving element provided in the surgical instrument.

As illustrated in FIG. 26, the computing device may attempt to decide target state information for a specific time (stage 2541p), decide whether the target posture exceeds the driving limit for the at least one driving element included in the surgical instrument, and decide a modified state constrained within the driving limit as target state information for the driving element when the driving limit is exceeded. For example, when the target posture exceeds the joint limit angle for at least one joint, a modified joint angle constrained within the joint limit angle may be decided as the target joint angle. On the other hand, when the target posture does not exceed the driving limit of the at least one driving element, information on the state of the driving element for achieving the target posture may be decided as target state information (stage 2545p). Accordingly, according to the method for driving the surgical instrument according to an embodiment of the present disclosure, even when the target posture according to the manipulation information exceeds the driving limit of the at least one driving element of the surgical instrument, an embodiment of the present disclosure may address the issue of interruption of surgical operation due to delay in algorithm calculation time.

Referring again to FIG. 25, the computing device may drive the at least one driving element provided in the surgical instrument according to the decided target state information (stage 2550). Herein, the computing device may update the reference posture of the user input interface with posture information after manipulating the user input interface in response to a decision that the target posture of the surgical instrument corresponding to the manipulation information of the user input interface exceeds the driving limit for the at least one driving element provided in the surgical instrument.

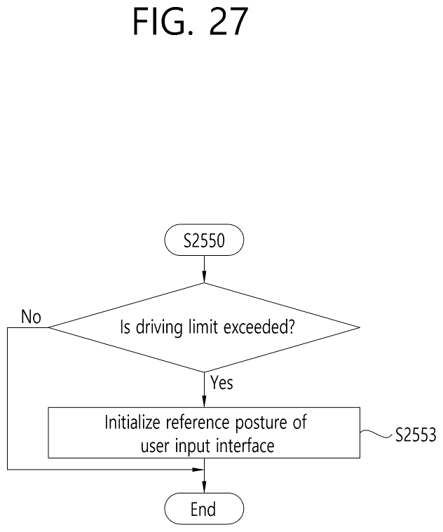

In this regard, FIG. 27 shows a conceptual information processing procedure of a reference posture initialization procedure when the driving limit of FIG. 25 is reached. As illustrated in FIG. 27, depending on whether the driving limit is exceeded and a constraint within the driving limit occurs, the computing device may initialize the reference posture of the user input interface to the current state of the user input interface when a constraint has occurred (stage 2553).

For example, when the target posture decided according to the manipulation of the user input interface exceeds the joint limit angle of at least one joint provided in the surgical instrument, and the target joint angle is set to a joint angle constrained within the joint limit angle, and then the joints of the surgical instrument are controlled accordingly, the reference posture information of the user input interface may be initialized with the posture information of the user input interface after the manipulation of a user. Accordingly, as described above, when the driving element is constrained within the driving limit, the issue of unintuitive control occurrences caused by the discrepancy between the state of the surgical instrument and/or the user input interface recognized by a user and the unrestricted state based on the computing device may be addressed.

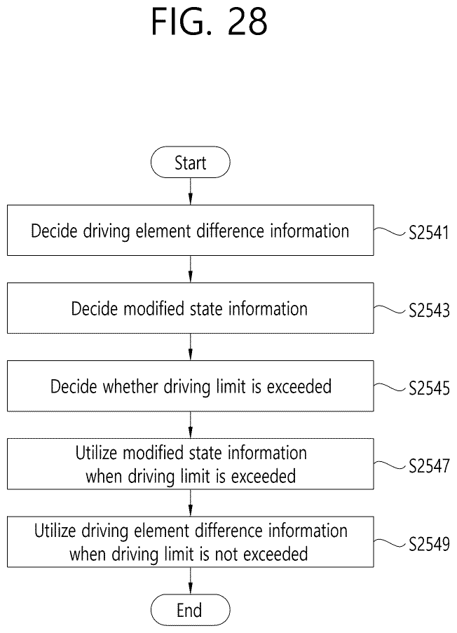

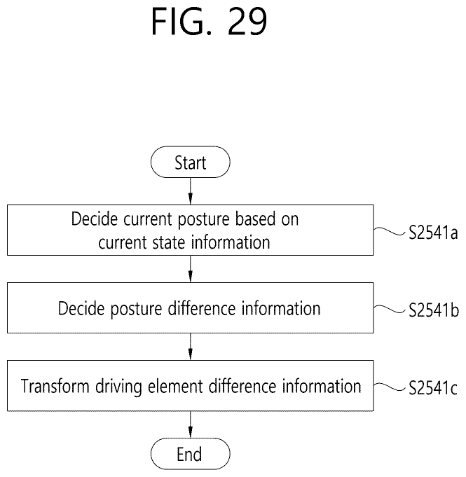

Hereinafter, a non-limiting but more detailed description will be given of the stage (stage 2540) for deciding target state information of the driving element provided in the surgical instrument based on whether the driving limit as illustrated in FIG. 25 is exceeded. In this regard, FIG. 28 is an exemplary detailed flowchart of the target state information decision stage of FIG. 26 according to an embodiment of the present disclosure. FIG. 29 is an exemplary detailed flowchart of the driving element difference information decision stage of FIG. 28.

As illustrated in FIG. 28, to decide the target state information of the driving element, the computing device may first decide driving the driving element difference information (stage 2541). The driving element difference information may reflect the degree of change in the state of the driving element required to change the surgical instrument to the target posture corresponding to the manipulation information. In other words, the driving element difference information may represent how much the at least one driving element of the surgical instrument needs to be changed to achieve the target posture. As a non-limiting example, when the driving element is a joint, the driving element difference information may be joint difference information and may indicate an amount of angle that needs to be changed by driving the joint.

To decide the driving element difference information (stage 2541), the computing device may begin performing an inverse kinematics transformation of the target pose as an input value. For the inverse kinematics transformation, the computing device may acquire current posture information of the surgical instrument, calculate the posture difference information, and perform a process of transforming the driving element difference information.

More specifically, as illustrated in FIG. 29, the computing device may first decide the current posture of the surgical instrument based on the information on the current state of the driving element (stage 2541a). As a non-limiting example, when the driving element is a joint, the computing device extracts joint information from the surgical instrument. Since the joints of the surgical instrument are provided with motors and encoders, the position information of each motor may be known, and the information may be called joint information. For example, the information on the current state of the driving element may include joint information.

The computing device, for example, uses Equation 4 below to decide the current posture information (T_{curr}^{SR}) of the surgical instrument using the current joint information (g_{curr}^{SR}) and forward kinematics (FK) of the surgical instrument.

$${T}_{curr}^{SR} = FK ( {q}_{curr}^{SR} ) \qquad [ Equation 4 ]$$

Thereafter, the computing device may decide the posture difference information based on the difference between the target posture and the current posture (stage 2541b). In other words, the computing device may calculate the posture difference information (X_{diff}^{SR}), which is the difference between the target posture information of the surgical instrument and the current posture information. According to an aspect, target posture information and/or current posture information may be expressed in the form of a homogeneous transformation matrix. In this connection, for example, as described in Equation 5 below, the computing device may transform the matrix in the form of the homogeneous transformation matrix to the form of a screw (X) (f₂( )) and then calculate the posture difference information using a subtraction operation.

$${X}_{diff}^{SR} = {f}_{2} ( {T}^{SR} ) - {f}_{2} ( {T}_{curr}^{SR} ) \qquad [ Equation 5 ]$$

Referring again to FIG. 29, the computing device may transform the posture difference information into driving element difference information (stage 2541c). For example, the computing device may multiply the posture difference information by the Jacobian matrix to obtain the joint difference information (q_{diff}^{SR}) of the surgical instrument, as described in Equation 6 below. Herein, the Jacobian matrix is one of the physical quantities that express the kinematic information of a robot, and the ‘+’ operation means a pseudo inverse operation.

$${q}_{diff}^{SR} = {J}^{+} {X}_{diff}^{SR} \qquad [ Equation 6 ]$$

Through this exemplary procedure, the computing device may decide the driving element difference information (stage 2541).

Referring again to FIG. 28, the computing device may decide the modified state information in which the driving result of the driving element is constrained to a range within the driving limit of the driving element (stage 2543). For example, when the driving element is a joint, the modified state information may include the safe joint information. Here, the safe joint information may include an angle value adjusted so that the target joint angle is constrained within the joint limit angle even when the joint angle for achieving the target posture corresponding to the manipulation information exceeds the joint limit angle. According to an aspect, the modified state information may be decided so that the driving result of the driving element approaches the driving limit as the degree to which the target posture exceeds the driving limit increases. To this end, the computing device may decide the modified state information based on the driving element difference information, the upward driving limit value of the driving element, the downward driving limit value of the driving element, and the tangent function. Hereinafter, a non-limiting but more specific procedure for deciding the modified state information (for example, the safe joint information) is described.

According to an aspect, in the case in which at least one joint provided in the surgical instrument exceeds the joint limit angle during an inverse kinematics transformation process, the computing device may calculate the safe joint information in which the joint angle that is the driving target is limited to within the joint limit angle. To this end, the computing device may perform transformation to a fictive joint angle, Jacobian linkage function calculation to obtain fictive joint difference information, fictive compensation angle calculation to maintain joint limit angles, calculation of fictive joint angles reflecting joint limit angle constraints, and transformation to joint angles.

First, in order to constrain the joint angle to the limit value, the computing device may transform the joint angle that is interrupted at the limit value into the fictive joint angle (z^{SR}) so as to connect continuously even near the limit value. The transformation is, for example, as described in Equation 7 below, the i^{th} fictive joint angle (zᵢ^{SR}) may be performed using the i^{th} current joint angle (qᵢ^{SR}), the upward driving limit value of the i^{th} joint angle (qᵢ^{SR-U}) and the downward driving limit value of the i^{th} joint angle (qᵢ^{SR-L}).

$${z}_{i}^{SR} = tan ( \frac{π ( 2 {q}_{i}^{SR} - {q}_{i}^{SR - U} - {q}_{i}^{SR - L} )}{2 ( {q}_{i}^{SR - U} - {q}_{i}^{SR - L} )} ) \qquad [ Equation 7$$

Thereafter, the computing device may decide the fictive joint difference information (z_{diff}^{SR}). The process of obtaining the fictive joint difference information (z_{diff}^{SR}) is similar to the process of obtaining the joint difference information described above. The procedure for multiplying the posture difference information by the Jacobian matrix is the same as the process for deciding the joint difference information, but the pseudo-inverse matrix calculation of the Jacobian linkage matrix (dβ) is additionally required. The Jacobian linkage function is calculated based on Equation 8 below, for example, when a total number of joints in the surgical instrument is n.

$$β = [ \begin{matrix}\frac{\partial {β}_{1} ( {z}_{1} )}{\partial {z}_{1}} & 0 & … & 0 \\ 0 & \frac{\partial {β}_{2} ( {z}_{2} )}{\partial {z}_{2}} & … & 0 \\ ⋮ & ⋮ & ⋱ & ⋮ \\ 0 & 0 & … & \frac{\partial {β}_{n} ( {z}_{n} )}{\partial {z}_{n}}\end{matrix} ] \qquad [ Equation 8 ]$$

Herein, each element of the diagonal matrix is calculated as shown in Equation 9 below based on the i^{th} fictive joint angle (zᵢ^{SR}), the upward limit value of the it joint angle (qᵢ^{SR-U}), and the downward limit value of the i^{th} joint angle (qᵢ^{SR-L}).

$$\frac{\partial {β}_{i} ( {z}_{i} )}{\partial {z}_{i}} = \frac{{q}_{i}^{SR - U} - {q}_{i}^{S R - L}}{π} \frac{1}{1 + {( {z}_{i}^{SR} )}^{2}} \qquad [ Equation 9 ]$$

Thereafter, before calculating the fictive joint difference information, the computing device may calculate a fictive compensation angle (y) to be added to constrain a fictive joint to the limit value when the fictive joint exceeds the limit angle. When a total number of joints in the surgical instrument is n, the fictive compensation angle is an n-dimensional vector. In this connection, the i^{th} fictive compensation angle (yᵢ) is calculated as shown in Equation 10 below based on the i×i^{th} element of the Jacobian linkage matrix

$$( \frac{\partial {β}_{i} ( {z}_{i} )}{\partial {z}_{i}} ) ,$$

the i^{th} fictive joint angle (zᵢ^{SR}), and the i^{th} joint difference angle (q_{diff-i}^{SR}).

$${y}_{i} = \{ \begin{matrix}- {z}_{i}^{SR} & if \vert {z}_{i}^{SR} \vert > {γ}_{i} and {q}_{diff - i}^{SR} * {z}_{i}^{SR} < 0 \\ 0 & else\end{matrix} \qquad [ Equation 10 ]$$

Herein, the constant (γᵢ) used to determine the conditional sentence is calculated using the epsilon determination constant (ε₁) as shown in Equation 11 below. The epsilon determination constant may refer to a reference value for determining a value sufficiently close to 0 during computer calculations, and may be set to an arbitrary value. In the method for driving the surgical instrument according to an embodiment of the present disclosure, for example, the constant may be set to 1.0⁻⁹, without being limited thereto.

$${γ}_{i} = \sqrt{\frac{{q}_{i}^{SR - U} - {q}_{i}^{SR - L}}{ε {}_{1} * π} - 1} \qquad [ Equation 11 ]$$

The computing device may calculate the fictive joint difference information (z_{diff}^{SR}) reflecting the joint limit angle constraints. Herein, the fictive joint difference information is calculated as shown in Equation 12 below based on the pseudo-inverse matrix value of the Jacobian linkage matrix (dβ⁺), joint difference information (q_{diff}^{SR}), fictive compensation angle linkage matrix (J_{c}), and fictive compensation angle (y).

$${z}_{diff}^{SR} = d {β}^{+} {q}_{diff}^{SR} + {J}_{c} γ \qquad [ Equation 12 ]$$

Herein, the fictive compensation angle linkage matrix (J_{c}) is calculated as shown in Equation 13 below.

$${J}_{c} = [ \begin{matrix}{J}_{c - 1} & 0 & … & 0 \\ 0 & {J}_{c - 2} & … & 0 \\ ⋮ & ⋮ & ⋱ & ⋮ \\ 0 & 0 & … & {J}_{c - n}\end{matrix} ] \qquad [ Equation 13 ]$$

Each element of the diagonal matrix is calculated as shown in Equation 14 below utilizing the i×i^{th} element

$$( \frac{\partial {β}_{i} ( {z}_{i} )}{\partial {z}_{i}} )$$

of the Jacobian linkage matrix and a determination constant (ε₁).

$${J}_{c - i} = \{ \begin{matrix}1 & if \frac{\partial {β}_{i} ( {z}_{i} )}{\partial {z}_{i}} < {ε}_{1} \\ 0 & else\end{matrix} \qquad [ Equation 14 ]$$

Thereafter, the computing device may decide the safe joint information (q_{safe}^{SR}) constrained by the joint limit value based on the calculated fictive joint difference information. The safe i^{th} joint difference information (q_{safe-i}^{SR}) may be calculated as shown in Equation 15 below through the i^{th} fictive joint angle (zᵢ^{SR}), the i^{th} fictive joint difference angle (z_{diff-i}^{SR}), the upward driving limit value of the i^{th} joint angle (qᵢ^{SR-U}) and the downward driving limit value of the i^{th} joint angle (qᵢ^{SR-L}).

$${q}_{safe - i}^{SR} = \frac{{q}_{i}^{SR - U} - {q}_{i}^{SR - L}}{π} {tan}^{- 1} ( {z}_{i}^{SR} + {z}_{diff - i}^{SR} ) + \frac{{q}_{i}^{SR - U} + {q}_{i}^{SR - L}}{2} \qquad [ Equation 15 ]$$

Accordingly, the computing device may decide the modified state information (for example, the safe joint information) (stage 2543).

As illustrated in FIG. 28, once the inverse kinematics transformation is completed, the computing device may decide whether the target posture causes the driving limit for the at least one driving element to be exceeded based on the driving element difference information and the modified state information (stage 2545). According to an aspect, the computing device may decide whether the driving limit is exceeded based on whether the difference between the modified driving element difference information and the driving element difference information exceeds a first predetermined threshold. Herein, the modified driving element difference information may be decided based on the difference between the modified state information and information on the current state of the driving element, and the first threshold may be, for example, a difference determination constant.

As a non-limiting example, when the driving element is a joint, the computing device determines whether at least one joint provided in the surgical instrument is constrained to the joint limit angle. The determination means that a True value is returned in Equation 16 below utilizing the safe ith joint difference information (q_{safe,diff-i}^{SR}), ith joint difference information (q_{diff-i}^{SR}), and difference determination constant (ε₂). The safe joint difference information may represent the difference between the current angle of the target joint and the safe joint angle. The difference determination constant is a reference value for determining whether a significant difference has occurred during computer calculations, and may be set to any value. As the value increases, the criterion for determining the joint limit angle constraint may increase. As the value decreases, the criterion for determining the joint limit angle constraint may decrease. In the method for deriving the surgical instrument according to an embodiment of the present disclosure, the difference determination constant may be set to, for example, 1.0⁻⁵, without being limited thereto.

$$\{ \begin{matrix}return True & if i exists such that \vert {q}_{diff - i}^{SR} - {q}_{safe , diff - i}^{SR} \vert > {ε}_{2} \\ return False & else\end{matrix} \qquad [ Equation 16 ]$$

In other words, the computing device may decide the driving element difference information to achieve the target posture corresponding to the manipulation information regardless of whether the driving limit is constrained (stage 2541). Even when the driving limit has been exceeded, the computing device may decide the modified state information that will have a value constrained within the driving limit (stage 2543). Thereafter, the computing device may calculate a value for the difference between the degree of state change of the driving element according to the driving element difference information and the degree of state change of the driving element according to the modified state information. When the difference value exceeds a predetermined threshold, the computing device may decide that the driving element is constrained within the driving limit (stage 2545).

Referring again to FIG. 28, the computing device may finalize the target state information using at least one of the modified state information or the driving element difference information depending on whether the driving limit is exceeded (stages 2547 to 2549). For example, the computing device may decide the modified state information as the target state information in response to a decision that the driving limit is exceeded (stage 2547), or may decide a value of adding the driving element difference information to the information on the current state of the driving element as the target state information in response to a decision that the driving limit is not exceeded and a decision that the driving element difference information is less than a second threshold (stage 2549). Accordingly, the computing device may repeatedly perform the procedure of deciding the target state information (stage 2540) until a decision is made that the driving limit is exceeded or a decision is made that the driving element difference information is less than the second threshold. Herein, the second threshold may be, for example, the epsilon determination constant.

As a non-limiting example, when the driving element is a joint, the computing device may calculate both the joint difference information and the safe joint information and then generate the target joint angle depending on whether the joint is constrained to a limit angle, or may generate the target joint angle after determining the size of the joint difference information.

For example, when it is decided that the joint is constrained to the limit angle (for example, when the result of Equation 16 is True), the final target state information, for example, the transformed joint information (gᵣₑₛ^{SR}), as described in Equation 17 below, may be updated with the safe joint information (q_{safe}^{SR}).

$${q}_{res}^{SR} = {q}_{safe}^{SR} \qquad [ Equation 17 ]$$

In other words, when the joint is constrained to the joint limit angle, the safe joint difference information with a value for the angle constrained within the joint limit is ultimately decided as the target joint angle.

When it is decided that the joint is not constrained to the limit angle (the result of Equation 16 is False), the computing device determines the magnitude of the joint difference information. The determination of the size of joint difference information may be calculated as shown in Equation 18 below using the epsilon determination constant (ε₁). The epsilon determination constant is a reference value for determining a value sufficiently close to 0 during computer calculations, and may be set to an arbitrary value. In the method for driving the surgical instrument according to an embodiment of the present disclosure, the epsilon determination constant may be set to, for example, 1.0⁻⁹, without being limited thereto.

$$\{ \begin{matrix}return True & if \vert {q}_{diff}^{SR} \vert > {ε}_{1} \\ return False & else\end{matrix} \qquad [ Equation 18 ]$$

Herein, when the size of the joint difference information is sufficiently small, the computing device updates the transformed joint information (qᵣₑₛ^{SR}) through the joint difference information (q_{diff}^{SR}) and the current joint information (q_{curr}^{SR}), as shown in Equation 19 below. When the joint difference information is not small enough, the computing device adds the joint difference information (q_{diff}^{SR}) to the current joint information (g_{curr}^{SR}) as shown in Equation 20 below, and then repeatedly performs the target state information decision stage (stage 2540).

That is, if the size of the joint difference information is sufficiently small, the transformed joint information is calculated as shown in Equation 19 below.

In other words, when the size of the joint difference information is sufficiently small, the transformed joint information is calculated as shown in Equation 19 below.

$${q}_{res}^{SR} = {q}_{curr}^{SR} + {q}_{diff}^{SR} \qquad [ Equation 19 ]$$

In addition, when the size of the joint difference information is not small enough, the current joint information is calculated as shown in Equation 20 below.

$${q}_{curr}^{SR} = {q}_{curr}^{SR} + {q}_{diff}^{SR} \qquad [ Equation 20 ]$$

Thereafter, the computing device may repeatedly perform the target state information decision stage (stage 2540).

In other words, the computing device may decide whether the target posture corresponding to the manipulation information exceeds the driving limit of the at least one driving element provided in the surgical instrument (stage 2545). When it is decided that the driving limit has been exceeded, the computing device may decide the modified status information as the final target state information (stage 2547). When the driving limit is not exceeded, the computing device may repeatedly perform a procedure of deciding the driving element difference information, deciding the modified state information, deciding whether the driving limit is exceeded, and deciding the target state information accordingly until the driving element difference information becomes smaller than a second threshold. Thereby, the surgical instrument may be driven sufficiently close to the target posture. Simultaneously, even when the at least one driving element provided in the surgical instrument reaches the driving limit, the issue of interruption of surgical operation due to delay in algorithm calculation time may be addressed.

In addition, as described above, the computing device may drive the surgical instrument with the decided target state information, for example, the transformed joint information (qᵣₑₛ^{SR}). Herein, when the at least one driving element is constrained within the driving limit, the reference posture information (T₀^{MC}) of the user input interface may be initialized. Accordingly, it is possible to address the issue of the surgical robot operating differently from the intuitive intention of a user while constraining the driving elements within the driving limits.

The aforementioned method may be implemented in a general-purpose digital computer that may be embodied as a program that may be executed by a computer and operates the program using a computer-readable recording medium. In addition, the data structure used in the aforementioned method may be recorded on a computer-readable recording medium through various members. The computer-readable recording media includes storage media such as magnetic storage media (for example, a ROM, RAM, USB, floppy disk, hard disk, etc.) and optical reading media (for example, a CD-ROM or a DVD).

The aforementioned method may be included and provided in a computer program product. The computer program product may be traded between a seller and a buyer as a product. The computer program product may be distributed in the form of machine-readable storage medium (for example, a compact disc read only memory (CD-ROM)) or may be directly distributed (for example, download or upload) online through an application store (for example, a Play Store™) or between two user devices (for example, the smartphones). In the case of online distribution, at least a portion of the computer program product may be temporarily stored or generated in a machine-readable storage medium such as a memory of a manufacturer's server, an application store's server, or a relay server.

Those skilled in the art related to the present embodiment will understand that the aforementioned description may be implemented in a modified form without departing from the essential characteristics. Therefore, the disclosed methods should be considered for purposes of description only and not for purposes of limitation. In addition, the scope of right is indicated in the claims, not the foregoing description, and should be interpreted to include all differences within the equivalent scope.

What is claimed is:

1. A method for driving a surgical instrument, the method comprising:
generating manipulation information of the surgical instrument based on an amount of change in a reference posture of a user input interface for controlling the surgical instrument;
determining a target posture of the surgical instrument, corresponding to the generated manipulation information;
generating target state information for at least one driving element based on whether the target posture exceeds a driving limit of the at least one driving element provided in the surgical instrument; and
driving the at least one driving element according to the target state information.

2. The method of claim 1, wherein:
the driving element comprises a joint;
the target state information comprises a target joint angle; and
the driving limit comprises a joint limit angle.

3. The method of claim 1, wherein the reference posture of the user input interface is configured to be updated with posture information before a manipulation of the user input interface before a first manipulation of the user input interface.

4. The method of claim 1, wherein the determining the target posture comprises:
determining the target posture based on a correlation between a predetermined movement of the user input interface and a movement of the surgical instrument.

5. The method of claim 1, wherein the generating the target state information comprises:
generating modified state information that constrains a driving status of the at least one driving element not to exceed the driving limit, in response to a determination that the target posture exceeds the driving limit of the at least one driving element provided in the surgical instrument.

6. The method of claim 5, wherein the modified state information is decided such that the driving result of the driving element approaches the driving limit as a degree to which the target posture exceeds the driving limit increases.

7. The method of claim 1, wherein the driving is configured to update the reference posture of the user input interface with posture information after a manipulation of the user input interface in response to a determination that the target posture exceeds the driving limit of the at least one driving element provided in the surgical instrument.

8. The method of claim 1, wherein the generating the target state information comprises:
generating driving element difference information based on a degree of state change of the at least one driving element, required to change the surgical instrument to the target posture;
generating modified state information in which a driving result of the at least one driving element is constrained to a range within the driving limit of the at least one driving element; and
determining whether the target posture exceeds the driving limit of the at least one driving element, based on the driving element difference information and the modified state information.

9. The method of claim 8, wherein the generating the driving element difference information comprises:
determining a current posture of the surgical instrument based on information on a current state of the at least one driving element;
generating posture difference information based on a difference between the target posture and the current posture of the surgical instrument; and
transforming the posture difference information into the driving element difference information.

10. The method of claim 8, wherein the generating the modified state information comprising:
generating the modified state information based on the driving element difference information, an upper limit of the driving limit of the at least one driving element, a lower limit of the driving limit of the at least one driving element, and a tangent function.

11. The method of claim 8, wherein the determining whether the target posture exceeds the driving limit comprises:
determining whether the target posture exceeds the driving limit based on whether a difference between modified driving element difference information and the driving element difference information exceeds a first predetermined threshold.

12. The method of claim 11, wherein the generating the modified driving element difference information comprises:
generating the modified driving element difference information based on a difference between the modified state information and information on a current state of the at least one driving element.

13. The method of claim 8, wherein the generating the target state information further comprises:
generating the modified state information as the target state information, in response to a determination that the driving limit is exceeded.

14. The method of claim 13, wherein the generating the target state information further comprises:
determining a value obtained by adding the driving element difference information to information on a current state of the driving element as the target state information, in response to a determination that the driving limit is not exceeded and a decision that the driving element difference information is less than a second threshold.

15. The method of claim 14, wherein the generating the target state information is repeatedly performed until a decision is made that the driving limit is exceeded or a decision is made that the driving element difference information is less than a second threshold.

16. An apparatus for driving a surgical instrument, the apparatus comprising:
at least one processor; and
at least one memory,
wherein the at least one processor is configured to:
generate manipulation information of the surgical instrument based on an amount of change in a reference posture of a user input interface for controlling the surgical instrument;
determine a target posture of the surgical instrument corresponding to the generated manipulation information;
generate target state information for at least one driving element based on whether the target posture exceeds a driving limit of the at least one driving element provided in the surgical instrument; and
drive the at least one driving element according to the target state information.

17. A surgical robot system, comprising:
a user input interface;
a surgical instrument; and
at least one processor,
wherein the at least one processor is configured to:
generate manipulation information of the surgical instrument based on an amount of change in a reference posture of a user input interface for controlling the surgical instrument;
determine a target posture of the surgical instrument corresponding to the generated manipulation information;
generate target state information for at least one driving element based on whether the target posture exceeds a driving limit of the at least one driving element provided in the surgical instrument; and
drive the at least one driving element according to the target state information.
